(19) United States
(12) Patent Application Publication
(10) Pub. No.: US 2011/0097423 A1
(43) Pub. Date: Apr. 28, 2011

(54) Gene Prognosis Predictor Signature for Colorectal Carcinoma

(75) Inventors: R. Daniel Beauchamp, Nashville, TN (US); Jesse J. Smith, Nashville, TN (US); Aixiang Jiang, Nashville, TN (US); Yu Shyr, Nashville, TN (US); Bing Zhang, Nashville, TN (US); Natasha G. Deane, Nashville, TN (US)

(73) Assignee: Vanderbilt University, Nashville, TN (US)

(21) Appl. No.: 12/909,620

(22) Filed: Oct. 21, 2010

Related U.S. Application Data
(60) Provisional application No. 61/254,045, filed on Oct. 22, 2009.

(51) Int. Cl. C12Q 1/68; G01N 33/68; C40B 30/00; A61K 33/24; A61P 35/00

(52) U.S. Cl. 424/649; 435/7.92; 436/501; 435/7.1; 435/6; 506/7

(57) ABSTRACT

The present invention is drawn to methods of assessing colorectal cancer prognosis by examining the expression of particular genes disregulated in this disease state. Subjects exhibiting disregulation in one or more of these genes will have a higher risk of cancer recurrence and death.

This application claims benefit of priority to U.S. Provisional Application Ser. No. 61/254,045, filed Oct. 22, 2009, the entire contents of which are hereby incorporated by reference.

This invention was made with government support under 5R21AG25466-2 awarded by the National Institutes of Health. The government has certain rights in the invention.

### BACKGROUND OF THE INVENTION

1. Field of the Invention

The present invention relates generally to the fields of biochemistry, molecular biology, and medicine. In certain aspects, the invention is related to to use of a panel of marker genes whose disregulated expression is prognostic for for colorectal cancer.

2. Description of Related Art

Colorectal carcinoma is the 3rd most commonly occurring non-cutaneous carcinoma and the 2nd leading cause of cancer-related death in the United States (Jemal et al., 2008). While it is well established that adjuvant chemotherapy for stage III colon cancer patients results in a survival benefit for the group, careful review of available data for this group reveals that 40-44% of stage III patients enrolled in “surgery-only” groups in clinical trials did not recur in five years even without adjuvant treatment (Ragnhammar et al., 2001). Furthermore, clinical trials have failed to demonstrate the benefit of adjuvant chemotherapy when applied to unselected patients with stage II colon cancer (Benson et al., 2004; Gill et al., 2004; Mamounas et al., 1999).

On the other hand, some studies suggest that a subset of high-risk stage II colon cancer patients may benefit from adjuvant therapy (Quasar Collaborative et al., 2007; Figueredo et al., 2008; Figueredo et al., 2004). Much of these data for stage II patients come from meta-analyses and the question of whether adjuvant treatment really improves outcomes in stage II patients with unproven “high-risk” features (e.g., T4 lesions (invasion of tumor through bowel wall), poorly differentiated histology or lymphovascular invasion) has not been addressed in a prospective clinical trial to date. Thus, an accurate and reliable method of identifying patients at greatest risk (e.g., “high-risk” stage II patients), who would benefit from systemic therapy, has the potential to improve outcomes within this patient group.

### SUMMARY OF THE INVENTION

Thus, in accordance with the present invention, there is provided a method of predicting prognosis in a human subject diagnosed with colorectal cancer comprising assessing expression of 3 or more of the following genes in a colorectal cancer sample obtained from said subject:
CXCR7, AK1, ACTB, MGP, HES1, TMEM14A, EGR1, VDR, C6orf64, NQO1, STOX2, ACPY2, SPRY4, DCTD, TACC2, PDLIM5, CRABP1, MMP13, MYOT, DFNB31, HPSE, TEX11, SYT17, MUM1L1, SLC25A30, CSN3, NMNAT3, DENND2A, CIRBP, SPDYA, S100A3, PRTN3, C20orf74 and HS3ST5;
   wherein increased expression of at least 2-fold of CXCR7, AK1, ACTB, MGP, HES1, TMEM14A, EGR1, VDR, C6orf64, NQO1, STOX2, ACPY2, SPRY4, DCTD, TACC2 or PDLIM5 as compared to expression observed in non-cancer cells, and/or decreased expression of at least 2-fold of CRABP1, MMP13, MYOT, DFNB31, HPSE, TEX11, SYT17, MUM1L1, SLC25A30, CSN3, NMNAT3, DENND2A, CIRBP, SPDYA, S100A3, PRTN3, C20orf74 or HS3ST5 as compared to expression observed in non-cancer cells, indicates a poor prognosis. The expression of at least one of MGP, PRTN3, TEX11, EGR1, HS3ST5, SPRY4, SLC25A30, C6orf64, PDLIM5, AK1 or DFNB31 may be analyzed.

The colorectal cancer may be stage I, II or III. The method may further comprising obtaining said colorectal cancer sample. The colorectal cancer may be colon cancer or rectal cancer. Assessing expression may comprise assessing protein expression, such as by ELISA, RIA, immunohistochemistry, or mass spectrometry. Assessing expression may alternatively comprise assessing mRNA expression, such as by quantitative RT-PCR, gene chip array expression, or Northern blotting. The expression observed in said non-cancer cell may be based on a pre-determined standard, or determined by assessing expression in a non-cancer cell from said subject.

Prognosis may comprises length of survival, such as disease-specific length of survival or overall survival. Prognosis may alternatively be length of time to recurrence. The method may further comprising making a treatment decision for said subject, such as to give chemotherapy to a subject having a poor prognosis as compared to median, or to not give chemotherapy to a subject having a favorable prognosis as compared to median. The method may further comprise treating said subject with adjuvant chemotherapy.

In a particular embodiment, MGP, PRTN3 and TEX11 are analyzed. Further, one may analyze one or more or all of EGR1, HS3ST5, SPRY4, SLC25A30, C6org64, PDLIM5, AK1, and/or DFNB31. The method may further comprise analyzing at least one further gene, including 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of said genes. In particular, MGP, PRTN3 and TEX11, EGR1, HS3ST5, SPRY4, SLC25A30, C6org64, and PDLIM5 are analyzed.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The following figures form part of the present specification and are included to further demonstrate certain aspects of the present invention. The invention may be better understood by reference to one or more of these figures in combination with the detailed description of specific embodiments presented herein.

FIGS. 1A-B—Cell Culture and Mouse Model. Murine model of metastasis, in vivo monitoring and ex vivo proof of metastases. (FIG. 1A) MC-38 parental cells (heterogeneous: blue and red) were subjected to six sequential passages through matrigel-coated transwells resulting in enrichment of invasive subpopulations of MC-38 cells (red). The population of cells resulting from six sequential passages was called “MC-38inv”. Following in vivo passaging, a stabilized cell line (pink cells) called “MC-38met” was established. (FIG. 1B) MC-38inv cells were tested alongside MC-38 parental cells for the ability to form lung metastasis in a tail vein assay. MC-38inv cells were significantly more metastatic compared with MC-38 parental cells in the tail vein assay as measured by bioluminescent imaging and formation of lung nodules (see Table 5, p<0.001).

FIGS. 2A-B—Recurrence signature development and functional genomic clustering analysis. (FIG. 2A) VMC two-step schematic for enrichment and informing of the 34-gene recurrence signature for colon cancer. Mouse genes were mapped to human orthologues and the set of 300 differentially expressed genes (MC-38 parental vs. MC-38met) was identified. These 300 genes were next refined with 19 “high-risk” patients from the VMC training dataset for concordance using an exact binomial test. This analysis revealed 34 genes with concordant expression among the 19 high-risk patients and the MC-38met cells. The 34-gene recurrence signature was then applied to the independent MCC database to determine whether it could be used to discriminate patients on the basis of outcomes. (FIG. 2B) Functional genomic clustering of the 34-gene recurrence signature. Cluster analysis of mean-centered gene expression data (rows) with individual VMC patients (columns) results in two distinct patient clusters (cluster 1/pink, cluster 2/green). Individual gene symbols in the signature are listed at the right side of the dendrogram and patient identifiers are listed along the bottom. The 19 VMC “high-risk” patients used in the concordance analysis are all marked with a red asterisk. The heatmap key is 4-fold on the original signal intensity scale, which corresponds to 2-fold on a log2 scale. Key: 1) Sample source: red=MC-38 parental; green=MC-38 invasive derivative (MC-38met); blue=patient samples; 2) Ensemble Human Gene identifiers: right side of heatmap (see Table 1 for more detailed information).

FIGS. 3A-B—Testing of the 34-gene recurrence signature in the Moffitt Cancer Center (MCC) dataset across all stages. Kaplan-Meier estimates of overall and disease-specific survival in the Moffitt test set across all stages are shown. Expression data for probes corresponding to the 34-gene recurrence signature were used to build the Cox proportional hazard model from patient data in the Vanderbilt dataset. Plots represent survival analyses in the MCC patient data set, based upon Beta and Wald statistics (see Methods section) from the Vanderbilt dataset. (FIG. 3A) Overall and (FIG. 3B) disease-specific survival analyses were performed.

FIGS. 4A-D—Kaplan-Meier estimates of survival using the putative recurrence score in stage II and III patients from the Moffitt Cancer Center (MCC) colon cancer test set. Kaplan-Meier estimates from 114 colon cancer (AJCC stages II and III) patients under study at MCC were analyzed using the 34-gene based recurrence score. Lower than median recurrence score is denoted in black and higher than median recurrence score is noted in red. A low recurrence score was associated with better disease-specific and disease-free survival in stage II patients: (FIG. 4A) cancer-related death: n=57 patients where high scores represent 9 of 9 total deaths; and (FIG. 4B) disease-free survival: n=55 where high scores represent 10 of 11 total events. Similarly, a low score was associated with better disease-specific and disease free survival in stage III patients: (FIG. 4C) cancer-related death: n=57 patients, where high scores represent 14 of 17 total deaths; and (FIG. 4D) disease-free survival: n=56, where high scores represent 16 of 20 total events. Five-year mortality and recurrence rates are shown for stage II and III patients (FIG. 4A-D).

FIG. 5—MC-38met gross and histopathology in tail vein and splenic assays. Gross and representative histological sections from lungs and livers of MC-38 parental (upper panel) and MC-38met injected (lower panel) mice are shown. For MC-38 parental-injected mice, minimal lung nodules (<10) and no liver metastases were noted. Prolific liver metastases (>200) were found In the MC-38met injected mice.

FIG. 6—Distribution of 10,000 permutation Wald tests of the 177 Moffitt Cancer Center patients with the 34-gene recurrence score. Beta and Wald statistics for each Affymetrix probe set were used along with expression data to build up a recurrence score for each patient. The score was used as the independent variable to perform overall survival analysis based on the Cox model. The Wald test P-value was saved as the observed P-value. For the re-sampling test, the inventors randomly chose 60 Affymetrix probe sets from the 54675 sets on the whole array. The inventors repeated the above procedure and generated one re-sampling Wald test P-value from the overall Cox model survival analysis. The inventors repeated the resampling and survival analysis procedure 10,000 times, generating 10,000 re-sampling Wald test P-values. The inventors transformed both the observed and re-sampling P-values into log₁₀ format, plotted a histogram of the 10,000 re-sampling log₁₀ (P-values), and added the observed log₁₀ (P-value).

FIG. 7—Cox model hazard ratios. Percentiles across high-score. Moffitt Cancer Center patients were plotted related to relative risk. Hazard ratios for the 50^{th}, 75^{th} and 90^{th} percentiles are plotted as compared to the 10^{th} percentile and show a 2.0, 3.1 and 4.6 increased risk of cancer-related death from the 50^{th} percentile upward.

FIGS. 8A-F—Detection of 34-gene elements by quantitative nuclease protection assay (qNPA®). qNPA® is a sensitive, hybridization based method for quantification of specific nucleic acids in complex mixtures. We used qNPA® to validate several elements of the 34-gene signature in representative colon cancer specimens with known metastasis scores (“hi”, “med”, or “lo/low”). Specific gene elements measured are listed below each graph. FIGS. 8A-C show results from elements predicted to be upregulated in patients with high metastasis score. FIGS. 8D-F show results from elements predicted to downregulated in patients with high metastasis scores. Statistical results from ANOVA with Bonferroni correction are shown.

### DESCRIPTION OF ILLUSTRATIVE EMBODIMENTS

As stated above, colorectal carcinoma is a widely prevalent form of cancer and cancer-related death in the United States (Jemal et al., 2008). Treatment of colorectal carcinoma typically involves surgery, optionally combined with adjuvant chemotherapy. However, for many patients, the surgery-only approach is just as effective. Thus, an accurate and reliable method of identifying patients at greatest risk (e.g., “high-risk” patients) who would benefit from systemic therapy has the potential to improve outcomes within this patient group.

The inventors utilized an experimental mouse model of metastasis to develop a gene expression profile that discriminated high versus low risk of cancer recurrence and death in patients with colon cancer. This biologically-based model identified distinct subsets of stage II and III colon cancer patients at greater risk of cancer recurrence and death. Using a recurrence score derived from the gene expression profile, the inventors found that stage III patients with a low recurrence score did not gain significant benefit from adjuvant chemotherapy, suggesting that these patients could have been spared the potentially toxic and costly effects of these treatments. Conversely, high recurrence score stage III patients treated with adjuvant chemotherapy had markedly improved outcomes compared with high recurrence score patients who did not receive adjuvant chemotherapy.

Based on these results, the inventors now propose the use this biologically-based gene expression profile as useful platform to facilitate selection of colon cancer patients who may benefit from adjuvant systemic therapy. By identifying the appropriate markers in samples obtained from colorectal cancer patients, one can identify those patients who should, or should not, receive following chemotherapy after surgical tumor resection. This will improve the efficiency and cost of treatments, and avoid unnecessary toxic side effects in subjects that will receive little benefit from chemotherapy. These and other aspects of the invention are discussed in detail below.

### I. COLORECTAL CANCER MARKERS

A. Detection Methods

It is within the general scope of the present invention to provide methods for the detection of mRNA and proteins, in particular for those genes listed in Table 1, below. Any method of detection known to one of skill in the art falls within the general scope of the present invention.

1. Nucleic Acid Detection

Nucleic acid sequences disclosed herein will find use in detecting expression of target genes, e.g., as probes or primers for embodiments involving nucleic acid hybridization. As used in this application, the term “polynucleotide” refers to a nucleic acid molecule that has been isolated essentially or substantially free of total genomic nucleic acid to permit hybridization and amplification, but is not limited to such. An oligonucleotide refers to a nucleic acid molecule that is complementary or identical to at least 5 contiguous nucleotides of a given sequence.

It also is contemplated that a particular polypeptide from a given species may be represented by natural variants that have slightly different nucleic acid sequences but, nonetheless, encode the same protein. In this respect, the term “gene” is used for simplicity to refer to a functional protein, polypeptide, or peptide-encoding unit. As will be understood by those in the art, this functional term includes genomic sequences, cDNA sequences, and smaller engineered gene segments that express, or may be adapted to express, proteins, polypeptides, domains, peptides, fusion proteins, and mutants.

A nucleic acid may be of the following lengths: about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs.

a. Hybridization

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772 and U.S. Patent Publication 2008/0009439. The relevant portions of these and other references identified in this section of the Specification are incorporated herein by reference.

b. In situ Hybridization

In situ hybridization (ISH) is a type of hybridization that uses a labeled complementary DNA or RNA strand (i.e., probe) to localize a specific DNA or RNA sequence in a portion or section of tissue (in situ), or, if the tissue is small enough (e.g. plant seeds, Drosophila embryos), in the entire tissue (whole mount ISH). This is distinct from immunohistochemistry, which localizes proteins in tissue sections. Fluorescent DNA ISH (FISH) can, for example, be used in medical diagnostics to assess chromosomal integrity. RNA ISH (hybridization histochemistry) is used to measure and localize mRNAs and other transcripts within tissue sections or whole mounts.

For hybridization histochemistry, sample cells and tissues are usually treated to fix the target transcripts in place and to increase access of the probe. As noted above, the probe is either a labeled complementary DNA or, now most commonly, a complementary RNA (riboprobe). The probe hybridizes to the target sequence at elevated temperature, and then the excess probe is washed away (after prior hydrolysis using RNase in the case of unhybridized, excess RNA probe). Solution parameters such as temperature, salt and/or detergent concentration can be manipulated to remove any non-identical interactions (i.e., only exact sequence matches will remain bound). Then, the probe that was labeled with either radio-, fluorescent- or antigen-labeled bases (e.g., digoxigenin) is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

c. Amplification of Nucleic Acids

Nucleic acids used as a template for amplification may be isolated from cells, tissues or other samples according to standard methodologies (Sambrook et al., 2001). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to any sequence corresponding to a nucleic acid sequence are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 2001). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Reverse transcription (RT) of RNA to cDNA followed by quantitative PCR (RT-PCR) can be used to determine the relative concentrations of specific mRNA species isolated from a cell. By determining that the concentration of a specific mRNA species varies, it is shown that the gene encoding the specific mRNA species is differentially expressed. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR amplification is directly proportional to the starting concentration of the target before the reaction began. By determining the concentration of the amplified products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can be determined for the respective tissues or cells. This direct proportionality between the concentration of the PCR products and the relative mRNA abundances is only true in the linear range of the PCR reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR for a collection of RNA populations is that the concentrations of the amplified PCR products must be sampled when the PCR reactions are in the linear portion of their curves.

A second condition for an RT-PCR experiment is to determine the relative abundances of a particular mRNA species. Typically, relative concentrations of the amplifiable cDNAs are normalized to some independent standard. The goal of an RT-PCR experiment is to determine the abundance of a particular mRNA species relative to the average abundance of all mRNA species in the sample.

Most protocols for competitive PCR utilize internal PCR standards that are approximately as abundant as the target. These strategies are effective if the products of the PCR amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

RT-PCR can be performed as a relative quantitative RT-PCR with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989).

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 2001). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 2001). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Various nucleic acid detection methods known in the art are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

d. Chip Technologies

Specifically contemplated by the present inventors are chip-based DNA technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al, 1991). It is contemplated that this technology may be used in conjunction with evaluating the expression level of a gene target.

e. Nucleic Acid Arrays

The present invention may involve the use of arrays or data generated from an array. Data may be readily available. An array generally refers to ordered macroarrays or microarrays of nucleic acid molecules (probes) that are fully or nearly complementary or identical to a plurality of mRNA molecules or cDNA molecules and that are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g., up to about 100 or even 1000 per square centimeter. Microarrays typically use coated glass as the solid support, in contrast to the nitrocellulose-based material of filter arrays. By having an ordered array of complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass and silicon Such arrays may vary in a number of different ways, including average probe length, sequence or types of probes, nature of bond between the probe and the array surface, e.g., covalent or non-covalent, and the like. The labeling and screening methods of the present invention and the arrays are not limited in its utility with respect to any parameter except that the probes detect expression levels; consequently, methods and compositions may be used with a variety of different types of genes.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610;287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 100 or more different probes. It is contemplated that they may contain 1000, 16,000, 65,000, 250,000 or 1,000,000 or more different probes. The probes can be directed to targets in one or more different organisms. The oligonucleotide probes range from 5 to 50, 5 to 45, 10 to 40, or 15 to 40 nucleotides in length in some embodiments. In certain embodiments, the oligonucleotide probes are 20 to 25 nucleotides in length.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm². The surface area of the array can be about or less than about 1, 1.6, 2, 3, 4, 5, 6, 7, 8, 9, or 10 cm².

Moreover, a person of ordinary skill in the art could readily analyze data generated using an array. Such protocols are disclosed above, and include information found in WO 9743450; WO 03023058; WO 03022421; WO 03029485; WO 03067217; WO 03066906; WO 03076928; WO 03093810; WO 03100448A1, all of which are specifically incorporated by reference.

2. Protein Detection

In certain embodiments, the present invention concerns determining the expression level of a protein corresponding to a target gene. As used herein, a “protein,” “proteinaceous molecule,” “proteinaceous composition,” “proteinaceous compound,” “proteinaceous chain” or “proteinaceous material” generally refers, but is not limited to, a protein of greater than about 200 amino acids or the full length endogenous sequence translated from a gene; a polypeptide of greater than about 100 amino acids; and/or a peptide of from about 3 to about 100 amino acids. All the “proteinaceous” terms described above may be used interchangeably herein.

In certain embodiments, the proteinaceous composition may be identified using an antibody. As used herein, the term “antibody” is intended to refer broadly to any immunologic binding agent such as IgG, IgM, IgA, IgD and IgE. The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (see, e.g., Harlow et al., 1988; incorporated herein by reference).

a. Proteinaceous Compositions

As used herein, an “amino molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties. Accordingly, the term “proteinaceous composition” encompasses amino molecule sequences comprising at least one of the 20 common amino acids in naturally synthesized proteins, or at least one modified or unusual amino acid.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. In certain embodiments a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur. Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (e.g., alter pH, ionic strength, and temperature).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that by itself does not adsorb molecules to any significant extent and that has a broad range of chemical, physical and thermal stability. The ligand should be coupled in such a way as to not affect its binding properties. The ligand also should provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

b. Immunodetection Methods

In some embodiments, the present invention concerns immunodetection methods. Immunodetection methods include enzyme linked immunosorbent assay (ELISA), radioimmunoassay (RIA), immunoradiometric assay, fluoroimmunoassay, chemiluminescent assay, bioluminescent assay, and Western blot, though several others are well known to those of ordinary skill. The steps of various useful immunodetection methods have been described in the scientific literature, such as, e.g., Doolittle et al. (1999); Gulbis et al. (1993); De Jager et al. (1993); and Nakamura et al. (1987), each incorporated herein by reference.

In general, the immunobinding methods include obtaining a sample containing a protein, polypeptide and/or peptide, and contacting the sample with a first antibody, monoclonal or polyclonal, in accordance with the present invention, as the case may be, under conditions effective to allow the formation of immunocomplexes.

These methods include methods for purifying a protein, polypeptide and/or peptide from organelle, cell, tissue or organism's samples. In these instances, the antibody removes the antigenic protein, polypeptide and/or peptide component from a sample. The antibody will preferably be linked to a solid support, such as in the form of a column matrix, and the sample suspected of containing the protein, polypeptide and/or peptide antigenic component will be applied to the immobilized antibody. The unwanted components will be washed from the column, leaving the antigen immunocomplexed to the immobilized antibody to be eluted.

The immunobinding methods also include methods for detecting and quantifying the amount of an antigen component in a sample and the detection and quantification of any immune complexes formed during the binding process. Here, one would obtain a sample suspected of containing an antigen or antigenic domain, and contact the sample with an antibody against the antigen or antigenic domain, and then detect and quantify the amount of immune complexes formed under the specific conditions.

In terms of antigen detection, the biological sample analyzed may be any sample that is suspected of containing an antigen or antigenic domain, such as, for example, a tissue section or specimen, a homogenized tissue extract, a cell, an organelle, separated and/or purified forms of any of the above antigen-containing compositions, or even any biological fluid that comes into contact with the cell or tissue, including blood and/or serum.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection designed by Charles Cantor uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

c. ELISAs

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

In one exemplary ELISA, antibodies are immobilized onto a selected surface exhibiting protein affinity, such as a well in a polystyrene microtiter plate. Then, a test composition suspected of containing the antigen, such as a clinical sample, is added to the wells. After binding and/or washing to remove non-specifically bound immune complexes, the bound antigen may be detected. Detection is generally achieved by the addition of another antibody that is linked to a detectable label. This type of ELISA is a simple “sandwich ELISA.” Detection may also be achieved by the addition of a second antibody, followed by the addition of a third antibody that has binding affinity for the second antibody, with the third antibody being linked to a detectable label.

In another exemplary ELISA, the samples suspected of containing the antigen are immobilized onto the well surface and/or then contacted with antibodies. After binding and/or washing to remove non-specifically bound immune complexes, the bound anti-antibodies are detected. Where the initial antibodies are linked to a detectable label, the immune complexes may be detected directly. Again, the immune complexes may be detected using a second antibody that has binding affinity for the first antibody, with the second antibody being linked to a detectable label.

Another ELISA in which the antigens are immobilized, involves the use of antibody competition in the detection. In this ELISA, labeled antibodies against an antigen are added to the wells, allowed to bind, and/or detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. An example of a washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. This may be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

d. Immunohistochemistry

The antibodies of the present invention may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and/or is well known to those of skill in the art (Brown et al., 1990; Abbondanzo et al., 1990; Allred et al., 1990).

Immunohistochemistry or IHC refers to the process of localizing proteins in cells of a tissue section exploiting the principle of antibodies binding specifically to antigens in biological tissues. It takes its name from the roots “immuno,” in reference to antibodies used in the procedure, and “histo,” meaning tissue. Immunohistochemical staining is widely used in the diagnosis and treatment of cancer.

Visualising an antibody-antigen interaction can be accomplished in a number of ways. In the most common instance, an antibody is conjugated to an enzyme, such as peroxidase, that can catalyse a colour-producing reaction. Alternatively, the antibody can also be tagged to a fluorophore, such as FITC, rhodamine, Texas Red, Alexa Fluor, or DyLight Fluor. The latter method is of great use in confocal laser scanning microscopy, which is highly sensitive and can also be used to visualize interactions between multiple proteins.

Briefly, frozen-sections may be prepared by rehydrating 50 mg of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

There are two strategies used for the immmunohistochemical detection of antigens in tissue, the direct method and the indirect method. In both cases, the tissue is treated to rupture the membranes, usually by using a kind of detergent called Triton X-100.

The direct method is a one-step staining method, and involves a labeled antibody (e.g. FITC conjugated antiserum) reacting directly with the antigen in tissue sections. This technique utilizes only one antibody and the procedure is therefore simple and rapid. However, it can suffer problems with sensitivity due to little signal amplification and is in less common use than indirect methods.

The indirect method involves an unlabeled primary antibody (first layer) which reacts with tissue antigen, and a labeled secondary antibody (second layer) which reacts with the primary antibody. The secondary antibody must be against the IgG of the animal species in which the primary antibody has been raised. This method is more sensitive due to signal amplification through several secondary antibody reactions with different antigenic sites on the primary antibody. The second layer antibody can be labeled with a fluorescent dye or an enzyme.

In a common procedure, a biotinylated secondary antibody is coupled with streptavidin-horseradish peroxidase. This is reacted with 3,3′-Diaminobenzidine (DAB) to produce a brown staining wherever primary and secondary antibodies are attached in a process known as DAB staining. The reaction can be enhanced using nickel, producing a deep purple/gray staining.

The indirect method, aside from its greater sensitivity, also has the advantage that only a relatively small number of standard conjugated (labeled) secondary antibodies needs to be generated. For example, a labeled secondary antibody raised against rabbit IgG, which can be purchased “off the shelf,” is useful with any primary antibody raised in rabbit. With the direct method, it would be necessary to make custom labeled antibodies against every antigen of interest.

e. Protein Arrays

Protein array technology is discussed in detail in Pandey and Mann (2000) and MacBeath and Schreiber (2000), each of which is herein specifically incorporated by reference.

These arrays, typcially contain thousands of different proteins or antibodies spotted onto glass slides or immobilized in tiny wells, allow one to examine the biochemical activities and binding profiles of a large number of proteins at once. To examine protein interactions with such an array, a labeled protein is incubated with each of the target proteins immobilized on the slide, and then one determines which of the many proteins the labeled molecule binds. In certain embodiments such technology can be used to quantitate a number of proteins in a sample.

The basic construction of protein chips has some similarities to DNA chips, such as the use of a glass or plastic surface dotted with an array of molecules. These molecules can be DNA or antibodies that are designed to capture proteins. Defined quantities of proteins are immobilized on each spot, while retaining some activity of the protein. With fluorescent markers or other methods of detection revealing the spots that have captured these proteins, protein microarrays are being used as powerful tools in high-throughput proteomics and drug discovery.

The earliest and best-known protein chip is the ProteinChip by Ciphergen Biosystems Inc. (Fremont, Calif.). The ProteinChip is based on the surface-enhanced laser desorption and ionization (SELDI) process. Known proteins are analyzed using functional assays that are on the chip. For example, chip surfaces can contain enzymes, receptor proteins, or antibodies that enable researchers to conduct protein-protein interaction studies, ligand binding studies, or immunoassays. With state-of-the-art ion optic and laser optic technologies, the ProteinChip system detects proteins ranging from small peptides of less than 1000 Da up to proteins of 300 kDa and calculates the mass based on time-of-flight (TOF).

The ProteinChip biomarker system is the first protein biochip-based system that enables biomarker pattern recognition analysis to be done. This system allows researchers to address important clinical questions by investigating the proteome from a range of crude clinical samples (i.e., laser capture microdissected cells, biopsies, tissue, urine, and serum). The system also utilizes biomarker pattern software that automates pattern recognition-based statistical analysis methods to correlate protein expression patterns from clinical samples with disease phenotypes.

### II. TREATMENT OF CANCER

In some embodiments, the invention further provides treatment of colorectal cancer. One of skill in the art will be aware of many treatments and treatment combinations may be used, some but not all of which are described below.

A. Formulations and Routes for Administration to Patients

Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by intradermal, subcutaneous, intramuscular, intraperitoneal or intravenous injection. Such compositions would normally be administered as pharmaceutically acceptable compositions. Of particular interest is direct intratumoral administration, perfusion of a tumor, or administration local or regional to a tumor, for example, in the local or regional vasculature or lymphatic system, or in a resected tumor bed (e.g., post-operative catheter). For practically any tumor, systemic delivery also is contemplated. This will prove especially important for attacking microscopic or metastatic cancer.

The active compounds may also be administered as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The actual dosage amount of a composition of the present invention administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

“Treatment” and “treating” refer to administration or application of a therapeutic agent to a subject or performance of a procedure or modality on a subject for the purpose of obtaining a therapeutic benefit of a disease or health-related condition.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease.

A “disease” can be any pathological condition of a body part, an organ, or a system resulting from any cause, such as infection, genetic defect, and/or environmental stress.

“Prevention” and “preventing” are used according to their ordinary and plain meaning to mean “acting before” or such an act. In the context of a particular disease, those terms refer to administration or application of an agent, drug, or remedy to a subject or performance of a procedure or modality on a subject for the purpose of blocking the onset of a disease or health-related condition.

The subject can be a subject who is known or suspected of being free of a particular disease or health-related condition at the time the relevant preventive agent is administered. The subject, for example, can be a subject with no known disease or health-related condition (i.e., a healthy subject).

In additional embodiments of the invention, methods include identifying a patient in need of treatment. A patient may be identified, for example, based on taking a patient history or based on findings on clinical examination.

B. Cancer Treatments

1. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP 16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate and pharmaceutically acceptable salts, acids or derivatives of any of the above.

2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present invention may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and can be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of your internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, γ-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, β, and γ; IL-1, GM-CSF and TNF (Bukowski et al., 1998; Davidson et al., 1998; Hellstrand et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras et al., 1998; Hanibuchi et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989).

4. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Gene Therapy

In yet another embodiment, the gene therapy may be applied to the subject. Suitable genes included inducers of cellular proliferation, tumor suppressors, or regulators of programmed cell death.

6. RNA Interference (RNAi)

RNA interference (also referred to as “RNA-mediated interference” or RNAi) is a mechanism by which gene expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin and Avery et al., 1999; Montgomery et al., 1998; Sharp and Zamore, 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin and Avery et al., 1999; Montgomery et al., 1998; Sharp et al., 1999; Sharp and Zamore, 2000; Tabara et al., 1999). It is generally accepted that RNAi acts post-transcriptionally, targeting RNA transcripts for degradation. It appears that both nuclear and cytoplasmic RNA can be targeted (Bosher and Labouesse, 2000).

siRNAs must be designed so that they are specific and effective in suppressing the expression of the genes of interest. Methods of selecting the target sequences, i.e., those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998). siRNA are well known in the art. For example, siRNA and double-stranded RNA have been described in U.S. Pat. Nos. 6,506,559 and 6,573,099, as well as in U.S. Patent Applications 2003/0051263, 2003/0055020, 2004/0265839, 2002/0168707, 2003/0159161, and 2004/0064842, all of which are herein incorporated by reference in their entirety.

Several further modifications to siRNA sequences have been suggested in order to alter their stability or improve their effectiveness. It is suggested that synthetic complementary 21-mer RNAs having di-nucleotide overhangs (i.e., 19 complementary nucleotides+3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

dsRNA can be synthesized using well-described methods (Fire et al., 1998). Briefly, sense and antisense RNA are synthesized from DNA templates using T7 polymerase (MEGAscript, Ambion). After the synthesis is complete, the DNA template is digested with DnaseI and RNA purified by phenol/chloroform extraction and isopropanol precipitation. RNA size, purity and integrity are assayed on denaturing agarose gels. Sense and antisense RNA are diluted in potassium citrate buffer and annealed at 80° C. for 3 min to form dsRNA. As with the construction of DNA template libraries, a procedures may be used to aid this time intensive procedure. The sum of the individual dsRNA species is designated as a “dsRNA library.”

The making of siRNAs has been mainly through direct chemical synthesis; through processing of longer, double-stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single-stranded RNA-oligomers followed by the annealing of the two single-stranded oligomers into a double-stranded RNA. Methods of chemical synthesis are diverse. Non-limiting examples are provided in U.S. Pat. Nos. 5,889,136, 4,415,723, and 4,458,066, expressly incorporated herein by reference, and in Wincott et al. (1995).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25mer lengths with the endogenous nuclease complex that converts long dsRNAs to siRNAs in vivo. They do not describe or present data for synthesizing and using in vitro transcribed 21-25mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single-stranded RNA is enzymatically synthesized from the PCR products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

Several groups have developed expression vectors that continually express siRNAs in stably transfected mammalian cells (Brummelkamp et al., 2002; Lee et al., 2002; Paul et al., 2002; Sui et al., 2002; Yu et al., 2002). Some of these plasmids are engineered to express shRNAs lacking poly (A) tails (Brummelkamp et al., 2002; Paul et al., 2002; Yu et al., 2002). Transcription of shRNAs is initiated at a polymerase III (pol III) promoter and is believed to be terminated at position 2 of a 4-5-thymine transcription termination site. shRNAs are thought to fold into a stem-loop structure with 3′ UU-overhangs. Subsequently, the ends of these shRNAs are processed, converting the shRNAs into ˜21 nt siRNA-like molecules (Brummelkamp et al., 2002). The siRNA-like molecules can, in turn, bring about gene-specific silencing in the transfected mammalian cells.

7. Other Agents

It is contemplated that other agents may be used with the present invention. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

C. Dosage

The amount of therapeutic agent to be included in the compositions or applied in the methods set forth herein will be whatever amount is pharmaceutically effective and will depend upon a number of factors, including the identity and potency of the chosen therapeutic agent. One of ordinary skill in the art would be familiar with factors that are involved in determining a therapeutically effective dose of a particular agent. Thus, in this regards, the concentration of the therapeutic agent in the compositions set forth herein can be any concentration. In some particular embodiments, the total concentration of the drug is less than 10%. In more particular embodiments, the concentration of the drug is less than 5%. The therapeutic agent may be applied once or more than once. In non-limiting examples, the therapeutic agent is applied once a day, twice a day, three times a day, four times a day, six times a day, every two hours when awake, every four hours, every other day, once a week, and so forth. Treatment may be continued for any duration of time as determined by those of ordinary skill in the art.

### III. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

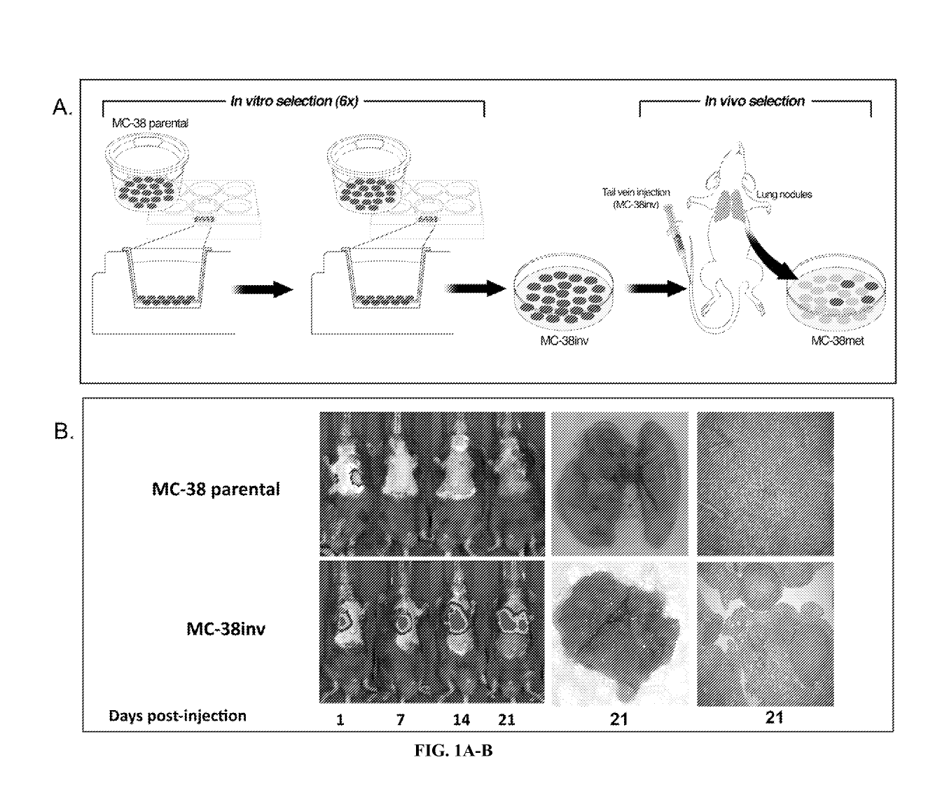

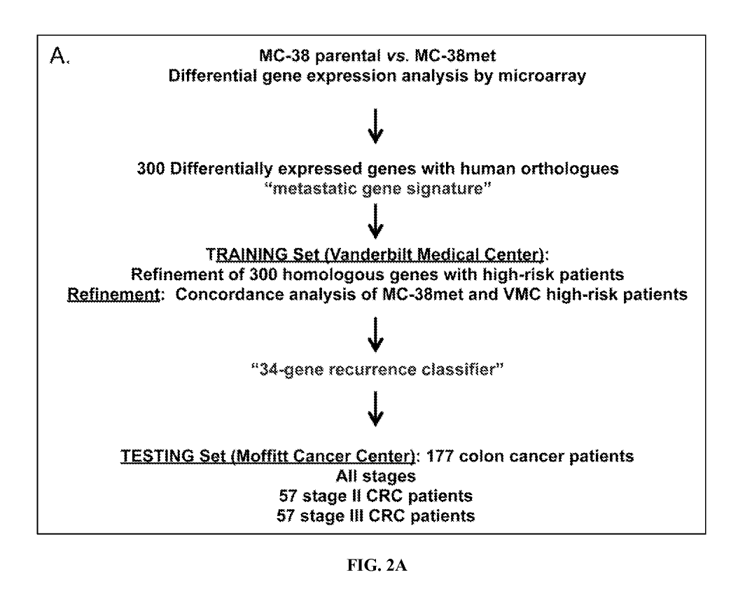

Cell Culture and Mouse Model. MC-38 mouse adenocarcinoma cells were obtained from the American Type Culture Collection (ATCC) and cultured (Lafreniere and Rosenberg, 1986). MC-38 cells were transfected with firefly luciferase gene in pGL3 basic (Promega, Madison, Wis.) and selected in 0.5 mg/mL G418 (Invitrogen, Carlsbad, Calif.). To enrich for invasive MC-38 cells, 7.5×10⁵ cells were seeded onto 6-well, 8.0 μM pore transwell polycarbonate membrane inserts (Costar, Cambridge, Mass.) coated with 2.5 mg/mL matrigel and incubated with serum-free DMEM in the upper chamber and complete DMEM in the bottom well. After 12 hours, invading cells were aseptically harvested by brief, gentle trypsinization and transferred to new dishes (Poste et al., 1981). Invading cells (Lafreniere and Rosenberg, 1986) were collected after six serial passages through matrigel-coated Boyden chambers (Poste et al., 1981). Cells collected after the 6^{th} passage were designated “MC-38inv” (FIG. 1). To quantify the enrichment of invasive cells, cells were again incubated in matrigel-coated transwell filters for 24 hours at 37° C., 5% CO2. Filters were washed and cells on the upper surface were removed with cotton swabs. The cells that had invaded were fixed in 4% paraformaldehyde for 10 minutes and stained with 1% crystal violet. Four random fields were counted to determine the numbers of invaded cells. To determine metastatic potential in vivo, equivalent numbers of MC-38 parental or MC-38inv cells were injected into the tail veins of C57BL/6 mice and the mice were followed for development of pulmonary metastases2. Briefly, unanesthetized mice (C57BL/6) were warmed with a heat lamp to allow for venous dilation. Mice were then placed into a plastic restraining apparatus and 2.5×10⁵ MC-38 parental, MC-38inv or MC-38met cells were injected via lateral tail vein (n=10). Successful injection and growth of metastatic lung nodules was confirmed by immediate and then weekly bioluminescence imaging (BLI). Bioluminescence imaging was carried out as previously described3,4. MC-38 in vivo metastatic cells (“MC-38met”) were derived from metastatic lung nodules following washing and sterilization of fresh tumor tissue in 0.04% sodium hypochlorite. The tissue was next rinsed in PBSA, minced into approximately 1 mm³ pieces, collected and resuspended in collagenase/neutral protease solution for 90 minutes at 37° C. with occasional shaking. Remaining tissue pieces were allowed to settle, and were then re-suspended in fresh collagenase/neutral protease solution and incubated at 4° C. overnight. The single-cell suspension was transferred to a new tube, centrifuged at 100 g for 5 minutes, washed once with growth medium and then re-suspended in growth medium and plated onto collagen-coated 25 cm flasks and 24-well dishes. Cells were grown at 37° C. with 5% CO₂ under G418 selection. The invasive phenotype of the MC-38met cells was confirmed both in vitro and in vivo (FIG. 1A and Table 6). For the splenic assay, 2.5×10⁵ cells were implanted under the splenic capsule 2-3 minutes prior to splenectomy and metastatic foci derived from resulting circulating tumor cells were followed as they formed in the liver by BLI. All animal work was carried out under protocols approved by the Vanderbilt Institutional Animal Care and Use Committee.

Tissue collection, processing and microarray platforms. The protocols and procedures for this study were approved by the Institutional Review Boards at the University of Alabama-Birmingham Medical Center, Vanderbilt Medical Center (VMC), the Veterans Administration Hospital (Nashville, Tenn.) and the H. Lee Moffitt Cancer Center (Tampa, Fla.). Informed consent was obtained for each patient. Representative sections of fresh tissue specimens from all surgically resected colon cancers and endoscopically-biopsied colorectal cancers were flash frozen in liquid nitrogen and stored at −80° C. until used for RNA isolation. Quality assessment slides were obtained to verify the diagnosis. RNA was purified using the RNeasy [symbol] kit from Qiagen (Valencia, Calif.) according to manufacturer's protocol. Samples from mouse and human sources were hybridized to Affymetrix Mouse Genome 430 2.0 GeneChip Expression and Affymetrix U133 Plus 2.0 GeneChip Expression Arrays, respectively.

Statistical Methods and analysis. The 300-gene metastasis-associated signature was determined using the limma package in Bioconductor (Smyth et al., 2005) based upon 3 criteria: (i)—fold change >2; (ii) False discovery rate (FDR) based on the moderated t-test followed by Benjamini and Hochberg's multiple test adjustment <0.01; and (iii) Log odds ratio of differential expression (B-statistic) >1. Directional concordance between MC-38met cells and 19 patients with poor outcome (17 stage IV patients and 2 stage III patients who developed metastatic recurrence) from VMC was determined using a cut-off of p≦0.10 (exact binomial test) to refine the metastasis-associated signature to the 34-gene “recurrence” signature. Integration of mouse and human microarray datasets was conducted as previously described (Lee and Thorgeirsson, 2004). Clustering based on Pearson's correlation coefficient was applied to the integrated data set.

Vanderbilt Cox Model (training dataset). The association between individual gene expression level and clinical endpoint (e.g., overall survival (OS) or disease-specific survival (DSS)), was first analyzed using a Cox Proportional Hazard (PH) model. Overall-survival is defined as death from any cause. Disease-specific survival is defined as cancer-related death. A disease-free survival event was defined as incidence of recurrence after R0 resection. Sixty Affymetrix probe sets can be found on the microarray HG U133 Plus 2.0 platform based on the 34 signature genes (e.g., multiple probes for each gene were found that mapped to each of the 34 genes). Their expression data were extracted from the VMC dataset (n=55). Expression data for each Affymetrix probe set were treated as the independent variable, and the Cox proportional hazard model was used for survival analyses. A compound score was next calculated for each patient by obtaining a Cox PH weighted sum (Wald score) of log-2 gene expression. The compound score for patient i is defined as [symbol] Wᵢ*Xᵢᵢ (where Wᵢ=Wald statistic score for gene i and Xᵢᵢ=log₂ gene i*expression level of patient i). Finally, the compound score was used to measure the impact of the 34-gene profile on survival and a censored c-index₇ was computed for the validation of the predictive value of the expression profile identified. The c-index is a probability of concordance between predicted and observed survival, with c=0.5 for random predictions, and c=1 for a perfectly discriminating model. The univariate analysis was completed using the compound scores to segregate patients into higher than median and lower than median compound score groups by Kaplan-Meier estimates to determine differences in survival by the log-rank statistic. The compound score, age gender, and grade were adjusted in the multivariate Cox model for overall, disease-specific and disease-free survival. The adjusted p-values as well as the adjusted 95% confidence intervals of the hazard ratios from the Cox model were reported.

Moffitt Cox Model (test dataset). Sixty Affymetrix probe sets can be found on the microarray HG U133 Plus 2.0 platform based on the 34 signature genes. Their expression data were extracted from the MCC dataset (n=177). Then we applied the estimated coefficients of the Cox PH model from the training set (n=55) to the MCC dataset (n=177).

Clinical Outcomes and testing of the 34-gene based recurrence score. The association between individual gene expression level and clinical endpoint (overall survival (OS), disease-specific survival (DSS) and disease-free survival (DFS)) were first analyzed using a Cox Proportional Hazard (PH) model (Tukey, 1993; Hedenfalk et al., 2001; Beer et al., 2002; Yanagisawa et al., 2003). A compound score was calculated for each patient by obtaining a Cox PH weighted sum (Wald score) of log-2 gene expression (Yanagisawa et al., 2003). The compound score was used to measure the impact of the 34-gene profile on survival (OS and DSS) and recurrence (DFS). A Vanderbilt-derived Cox model and re-sampling Wald tests were utilized to rule out over-fitting of the model. The estimated coefficients of the Cox PH model in the VMC data (55 patients) were applied to the MCC dataset (177 patients). In addition, a censored C-index (Harrell et al., 1982) was computed to validate the predictive value of the identified expression profile.

Univariate analysis (e.g., log-rank test) was completed using the compound scores to segregate patients into higher than median and lower than median compound score groups. The compound score, age, gender and tumor grade were adjusted in the multivariate Cox model for both DSS and DFS. Adjusted p-values as well as the adjusted 95% confidence intervals of the hazard ratios from the Cox model were reported. Fisher's Exact Test for a 2×3 table was used to determine differences between histology and recurrence score. Fisher's Exact Test was used for the stages II and III recurrence score, T4 and histological analyses as well as for the stage III recurrence score/chemotherapy analysis.

Ingenuity Pathways Analysis. Data were analyzed through the use of Ingenuity Pathways Analysis (IPA) and networks were generated (see Table 1) through the use of IPA (Ingenuity Systems, world-wide-web at ingenuity.com). A dataset containing gene identifiers and corresponding expression values was uploaded into the application. Each gene identifier was mapped to its corresponding gene object in the Ingenuity Pathways Knowledge Base. The cutoffs described in the statistical sections were used to identify genes whose expression was significantly differentially regulated. These genes, called focus genes in IPA, were overlaid onto a global molecular network developed from information contained in the Ingenuity Pathways Knowledge Base. Networks of these focus genes were then algorithmically generated based on their connectivity. The Functional Analysis identified the biological functions and/or disease that were most significant in the data set. Fisher's Exact Test was used to calculate a p-value determining the probability that each biological function and/or disease assigned to the data set is due to chance alone.

TABLE 1
Characterization of the 34-gene recurrence signatures

| Gene Symbol | -Fold Change | Processes/Networks |
|---|---|---|
| CXR7 | 3.9 | Cancer, survival/growth and chemotaxis |
| AK1 | 2.8 | Nucleotide binding |
| ACTB | 2.8 | Cancer, cell morphology and motility, growth, |
| | | polartization and adhesion |
| MGP | 2.8 | Cell-cell signaling, branching, migration |
| HES1 | 2.8 | Cancer, endrocrine function, cell death |
| TMEM14A | 2.6 | Cell proliferation (target of CREB) |
| EGR1 | 2.5 | Cancer, endocrine function, cell death |
| VDR | 2.4 | Cancer, endocrine function, cell death |
| C6orf64 | 2.4 | Membrane dynamics |
| NQ01 | 2.4 | Cancer, cell death |
| STOX2 | 2.3 | Putative stem cell marker |
| ACPY2 | 2.2 | Mutated in aromatic rice |
| SPRY4 | 2.1 | Cancer, cell migration, proliferation, differentiation |
| DCTD | 2.1 | Nucleotide biosynthesis |
| TACC2 | 2.1 | Cancer, biogenesis, morphology, proliferation |
| PDLIM5 | 2.0 | Cancer, actin binding |
| HS3ST5 | −2.0 | Putative epigenetic regulation |
| C20orf74 | −2.1 | GTPase regulation |
| PRTN3 | −2.1 | Cell-cell signaling, immune response |
| S100A3 | −2.2 | Cancer |
| SPDYA | −2.3 | Putative cell cycle |
| CIRBP | −2.3 | Cancer, nucleotide binding |
| DENND2A | −2.4 | Unknown |
| NMNAT3 | −2.4 | Nucleotide biosynthesis |
| CSN3 | −2.5 | Cell cycle, membrane dynamics |
| SLC25A30 | −2.6 | Oxidative stress |
| MUM1L1 | −2.8 | Unknown |
| SYT17 | −3.1 | Membrane protein |
| TEX11 | −3.7 | Cell cycle/division |
| HPSE | −4.7 | Cell-cell signaling, immune response |
| DFNB31 | −5.1 | Cell-cell signaling |
| MYOT | −7.1 | Cancer, actin filaments and stress fibers |
| MMP13 | −17.3 | Cell-cell signaling, immune response |
| CRABP1 | −21.0 | Cancer, CpG island methylation |

Genes that were upregulated or downregulated in both MC-38met derived cells and in 19 patients from VMC with poor-prognosis are outlined in table format. The gene symbols and associated fold-change in gene expression for MC-38 parental versus MC-38met as determined by microarray are given. Known relationships (connectedness) between upregulated and downregulated genes were determined independently and, where networks existed, functional enrichment within the networks were determined in Ingenuity Pathways Analysis (world-wide-web at ingenuity.com), PubMed and on the Affymetrix website (see Methods).

URLs. Complete Minimum information about a microarray experiment (MIAME)-compliant datasets for analysis are available at the world-wide-web at vicc.org/biostatistics/GeneSetting.php.

Accession codes. NCBI GEO: Gene expression microarray data will be submitted upon acceptance for publication.

Cross-species bioinformatics and differential expression analysis. Microarray data for the metastatic MC-38met cells and the parental MC-38 cells and the human data were processed using the Robust MultiChip Analysis₅ algorithm as implemented by Bioconductor (pmid:

12582260). Mouse probe set identifiers (IDs) were mapped to Ensembl Gene IDs based on the mapping provided by Ensembl V49 (world-wide-web at ensembl.org). Median expression levels from multiple probe sets corresponding to the same gene were calculated. Mouse genes with one-to-one human orthologue mapping as annotated by Ensembl V49 were carried forward for differential expression analysis using the limma package in Bioconductor6. The following three criteria were used to select genes that are differentially expressed between MC-38 parental and MC-38met cells based on results generated from the limma package: (i)—fold change >2; (ii) false discovery rate (FDR) based on the moderated t-test followed by Benjamini and Hochberg's multiple-test adjustment <0.01; and (iii) log odds ratio of differential expression (B statistic) >1. This analysis resulted in a subset of 300 differentially expressed genes that could be unambiguously mapped to human orthologues. Gene expression datasets were separately standardized such that each gene had a mean expression value of 0 and a standard deviation of 1 across samples in a dataset. A direction of expression was assigned for each gene in each sample based on the sign of the standardized value.

Metastasis-associated Gene Profile Development. Each gene of the murine training-set was examined in the VMC patient-derived training set in patients with stage III or stage IV disease who had experienced metastasis or cancer-related death. Nineteen patients fell into these categories and the median expression level of each of the 300 genes was examined for concordance with MC-38met median expression. The genes with concordance (+1 or −1) in at least 13 of 19 patients (Exact binomial test, p≦0.10) were selected for the putative recurrence profile. The concordance analysis resulted in a 34-gene profile that was termed the ‘34-gene recurrence score’ or more simply the ‘34-12 gene recurrence signature.’ See data posted at the world-wide-web at vicc.org/biostatistics/GeneSetting.php for files used for concordance analysis.

Clustering analysis of the 34-gene recurrence signature. Average linkage clustering based on Pearson's correlation coefficient was applied to the integrated data set. The ceiling was set to cover 95% of all data points (e.g., the top 5% z-scores were truncated). The corresponding z-score is 2.00 for the 34-gene signature.

Distribution of 10,000 re-sampling Wald tests in the test set with the 34-gene recurrence signature. Sixty Affymetrix probe sets can be found on the microarray HG U133 Plus 2.0 platform based on the 34 signature genes. Their expression data were extracted from the MCC dataset. Expression data for each Affymetrix probe set were treated as the independent variable, and the Cox proportional hazard model was used for survival analyses. Beta and Wald statistics for each Affymetrix probe set were used along with expression data to build up a compound score for each patient. The compound score was used as the independent variable to perform overall survival analysis based on the Cox model. The Wald test P value was saved as the observed P value. For the re-sampling test, the inventors randomly chose 60 Affymetrix probe sets from the 54675 sets on the whole array. The inventors repeated the above procedure and generated one re-sampling Wald test P value from the overall Cox model survival analysis. The inventors repeated the re-sampling and survival analysis procedure 10,000 times, generating 10,000 re-sampling Wald test P values. The inventors transformed both the observed and resampling P values into log₁₀ format, plotted a histogram of the 10,000 re-sampling log₁₀ (P values), and added the observed log₁₀ (P value).

Cox modeling of disease-specific survival in the test set for relative risk according to percentile score. Percentiles across high-score patients were plotted related to relative risk. Hazard ratios for 50^{th}, 75 ^{th} and 90 ^{th} percentiles were plotted as compared to the 10^{th} percentile.

### Example 2

### Results

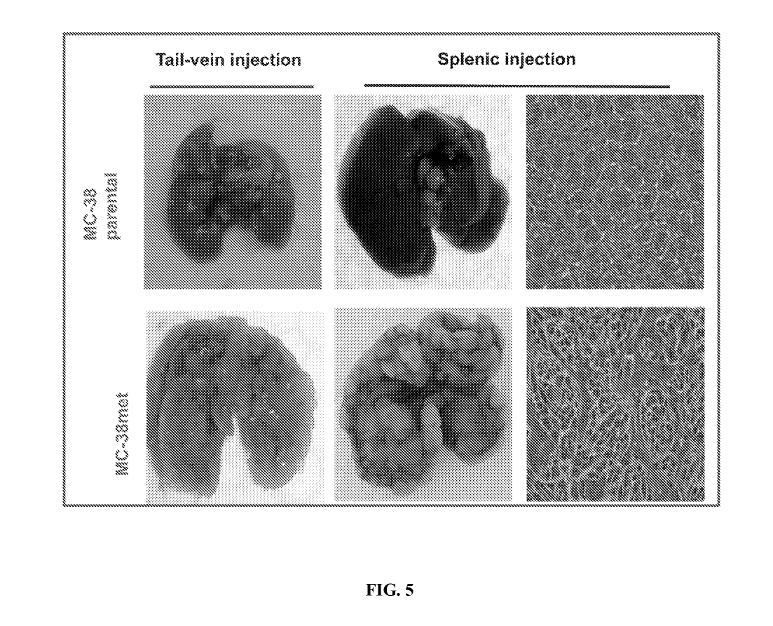

Development of an immunocompetent mouse model of colon cancer metastasis. Tumors are a heterogeneous mixture of cells with differing invasive and metastatic potential. Therefore, the inventors used a conventional invasion assay to enrich for a sub-population of highly invasive MC-38 mouse colon cancer cells (FIG. 1A; MC-38inv). Following six serial passages through matrigel, MC-38inv cells were 6-fold more invasive than MC-38 parental cells. In vivo, MC-38inv cells were significantly more metastatic to the lung as compared with MC-38 parental cells after tail vein injection (FIG. 1B; Table 5, p<0.001). Lung tumors derived from MC-38inv cells were cultured to derive a highly metastatic cell line, MC-38met. These MC-38met cells were injected into the tail vein and spleen and produced extensive metastatic tumors in the lung and liver respectively (see FIG. 5 and Table 6).

TABLE 5
Quantification of lung nodules from MC-38 parental and MC-38 inv cells

| | MC-38 Parental | MC-38 inv | |
|---|---|---|---|
| | (n = 10) | (n = 10) | p-value |
| Mean nodule # | 1.7 | 102.3 | <0.001 |
| (median, s.d.) | (1.0, 1.89) | (102.0, 24.7) | |

MC-38 parental and MC-38 inv cells, derived as described in FIG. 1 and Methods, were injected into the tail veins of C57BL6 mice. At 21 days post-injection, mice were sacrificed and lung nodules counted at necropsy. Summary statistics for the data and results from a Mann-Whitney test for statistical significance are outlined in the table (standard deviation (s.d.)).

TABLE 6
Hepatic and lung metastases (splenic and tail vein models) and
quantitated necropsy results

| | MC-38 parental | MC-38 met | |
|---|---|---|---|
| Hepatic Metastases | (n = 8) | (n = 6) | p-value |
| Mean liver weight in | 1.46 | 5.37 | 0.002 |
| Grams (median s.d.) | (1.45, 0.13) | (6.5, 2.27) | |
| Incidence (%) | 0/8 (0%) | 6/6 (100%) | <0.001 |
| | MC-38 parental | MC-38 met | |
| Lung Metastases | (n = 6) | (n = 5) | p-value |
| Mean lung weight in | 0.32 | 1.34 | 0.005 |
| Grams (median s.d.) | (0.30, 0.08) | (1.5, 0.37) | |

Quantification of the incidence of liver metastasis in the splenic assay and mean liver weights taken at necropsy with summary statistics is shown. Of note, in regard to MC-38 met liver metastasis, 2 mice died prematurely from massive liver metastasis and analysis was done on the 6 that survived to the end of the three-week experiment. Quantification of lung weights from the tail vein assay is also shown in the lower panel of the table. The Mann-Whitney test was used to calculate significance in SPSS (v.16) and summary statistics are displayed (standard deviation (s.d.)).

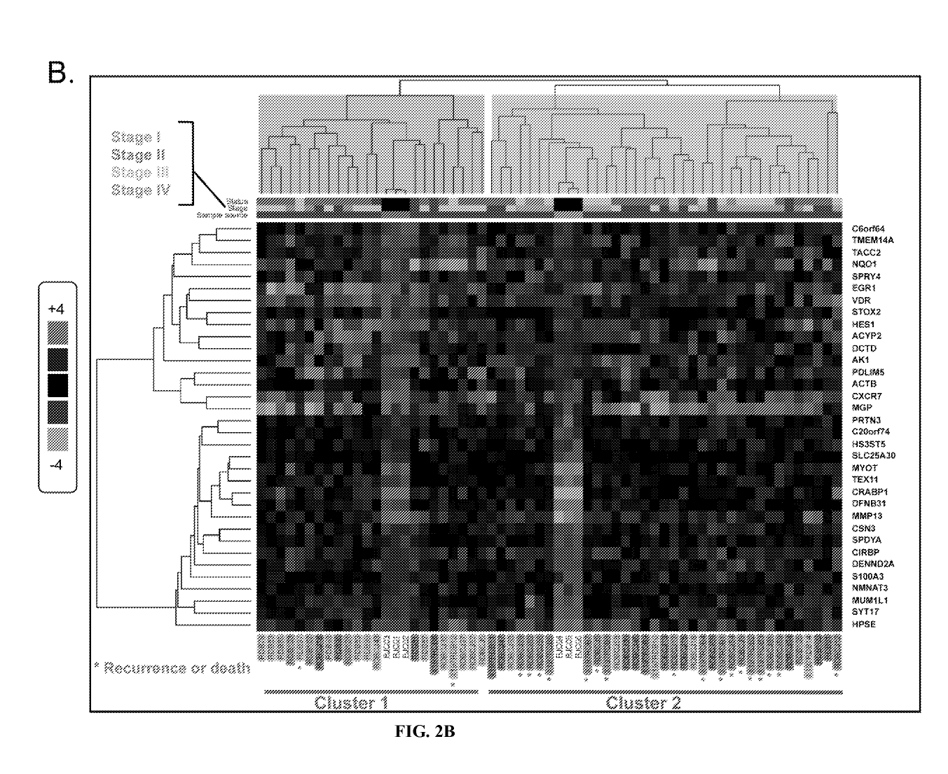

Discovery of a gene expression profile associated with metastasis: mouse to man. A flow diagram of the derivation of the metastatic gene expression signature and its refinement and testing is provided in FIG. 2A. MC-38 parental and MC-38met cell mRNA expression profiles were examined by microarray and compared to evaluate the gene expression changes associated with invasion and metastasis. Gene elements from this microarray profile were mapped to 11,465 corresponding human orthologues. Differential expression analysis identified 300 genes designating a “metastasis gene signature” from the MC-38met versus MC-38 parental comparison. The 300 genes were determined using the limma package in Bioconductor (Smyth et al., 2005) based upon -fold change >2, false discovery rate <0.01 and a log odds ratio of differential expression (B-statistic) >1.

In order to refine the signature with relevance to cancer recurrence, each gene from the metastatic signature was scored for directional concordance (see Methods) with gene expression data from 19 “high-risk” Vanderbilt Medical Center (VMC) colon cancer patients that either had metastatic disease or had died from cancer progression. Thirty-four genes (Table 1) from the metastasis gene signature exhibited directional concordance in 13 of the 19 “high-risk” patients. Since the 300 genes had been selected with stringent statistical criteria, the inventors did not further attempt to determine the minimum number of genes that could discriminate outcomes.

The resultant 34-gene expression pattern was designated the “recurrence gene signature.” The combined recurrence gene signature and the integrated VMC mouse/human microarray dataset was subjected to unsupervised cluster analysis. The recurrence gene signature separated patients into two clusters (FIG. 2B): MC-38 parental (cluster 1, pink) and MC-38met (cluster 2, green). The cluster associated with MC-38met cells contained 17 of the 19 “high-risk” patients (89.5%) used in the signature refinement process.

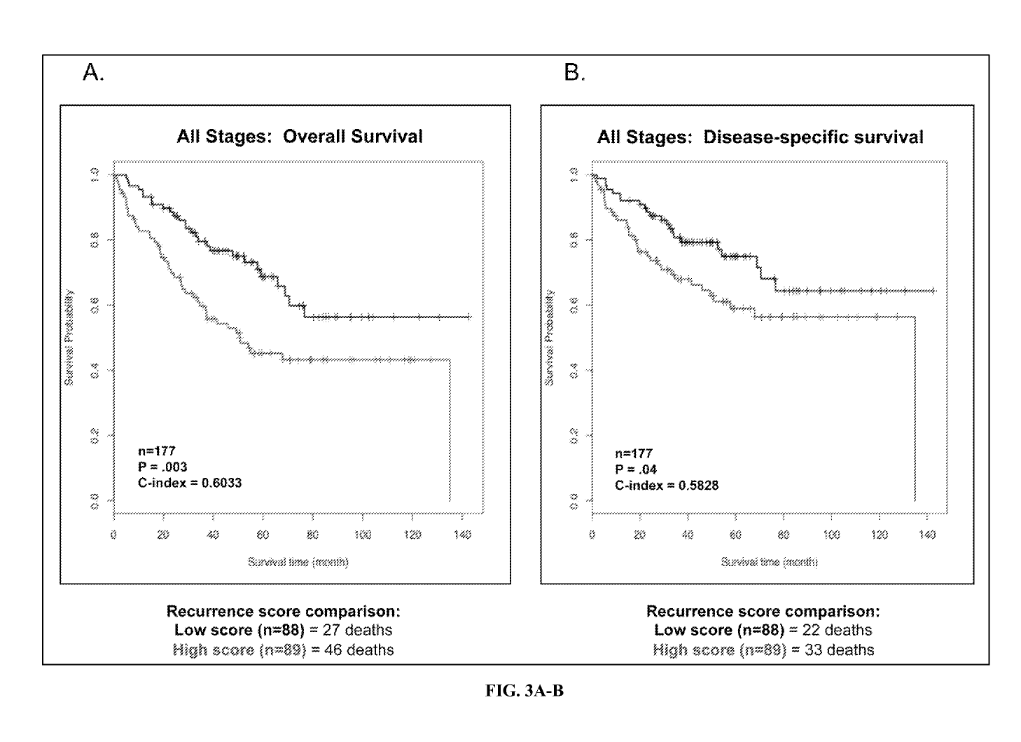

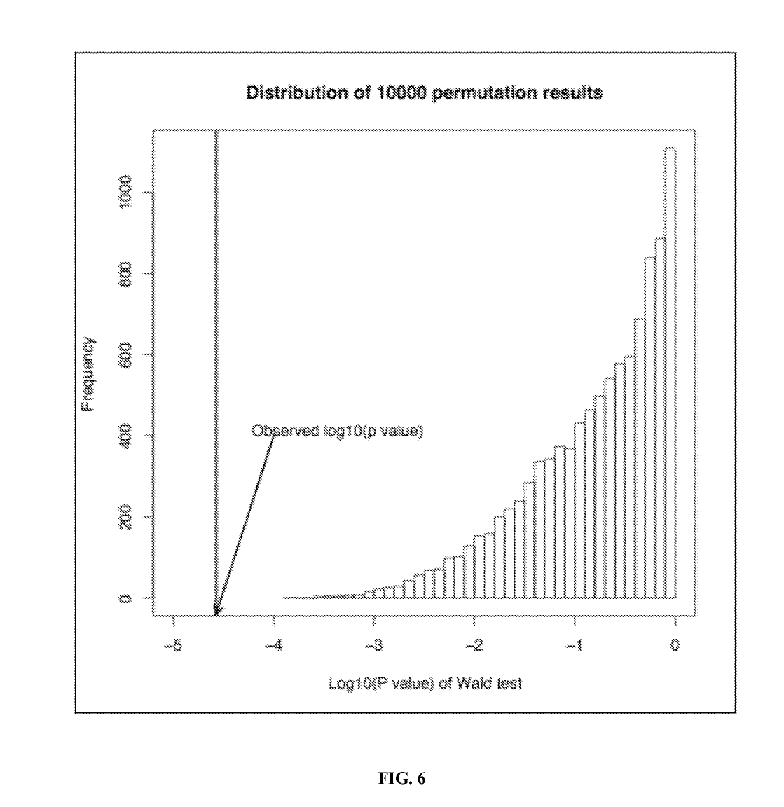

The recurrence signature identifies poor outcome colon cancer patients in an independent colon cancer dataset. An independent human colon cancer gene expression and clinical database from the H. Lee Moffitt Cancer Center (MCC) was used to test the ability of the recurrence signature to discriminate patients at increased risk of cancer recurrence and death. The demographics for the training (VMC) and test set (MCC) are shown in Table 2. Although 195 patients were available for analysis in the MCC group, we focused on colon cancer patients (n=177) to avoid potential confounding effects of neoadjuvant and radiation therapy in rectal cancer patient tumor samples and outcomes. A method for weighted scoring of the gene expression pattern for the recurrence signature was applied (see Methods) and recurrence “scores” were created. Patients were segregated into higher and lower than median recurrence score groups and survival analysis was performed between the two groups. To rule out over-fitting of the model, three separate statistical approaches were applied. First, the inventors developed the recurrence score based on the Cox Proportional Hazard (PH) model in the VMC data (55 patients) and then applied the estimated coefficients of the Cox PH model from VMC data to the MCC dataset (177 patients). The sign and magnitude of the coefficient was completely based on the training dataset (VMC) and then tested in the MCC dataset. High recurrence score colon cancer patients across all stages in the MCC dataset had significantly worse overall and disease-specific survival compared with low recurrence score patients (FIGS. 3A-B, p=0.003 and p=0.04 respectively). Second, multiple permutation testing was performed using the 34-gene recurrence signature and the recurrence score was also robust in this model (see FIG. 6). Third, the c-index was calculated to validate the predictive value of the gene expression profile recurrence score.

TABLE 2
Study demographics and case information

| Study Demographics | VMC Training | | MCC Training | |
|---|---|---|---|---|
| Sample size | 55 | | 177 | |
| Mean age (s.d.) | 62.3 | (14.1) | 65.5 | (13.1) |
| Sex (% male) | 30 | (54.5%) | 96 | (54.2%) |
| Stage I | 4 | (7.3%) | 24 | (13.6%) |
| Stage II | 15 | (27.3%) | 57 | (32.2%) |
| Stage III | 19 | (34.5%) | 57 | (32.2%) |
| Stage IV | 17 | (30.9%) | 39 | (22.0%) |
| Median follow-up | 50.2 | (0.4-111.3) | 48.1 | (0.92-142.6) |
| in months (min-max) | | | | |
| Deaths | 20 | (36.3%) | 73 | (41.2%) |
| Caucasian (%) | 50 | (90.9%) | 151 | (85.3%) |
| Black (%) | 4 | (7.3%) | 9 | (5.1%) |
| Other (%) | 1 | (1.8%) | 17 | (9.6%) |

Demographics and case information for the 55 patients (training dataset) represented by Vanderbilt Medical Center (VMC) and the 177 patients (testing dataset) represented by the Moffitt Cancer Center (MCC) are presented in table format. The Vanderbilt training set includes 14 patients from the University of Alabama-Birmingham Medical Center whose tumors were provided by M.J.H. All patients were diagnosed with colorectal adenocarcinoma (stages I-IV) according to current American Joint Commission on Cancer (AJCC) guidelines. 177 of 205 MCC patients met the criteria of having AJCC stage I-IV colon cancer as well as available grade, age and gender information. Other in the VMC medical record implies not otherwise specified and it implies Hispanic or not otherwise specified in the MCC medical record.

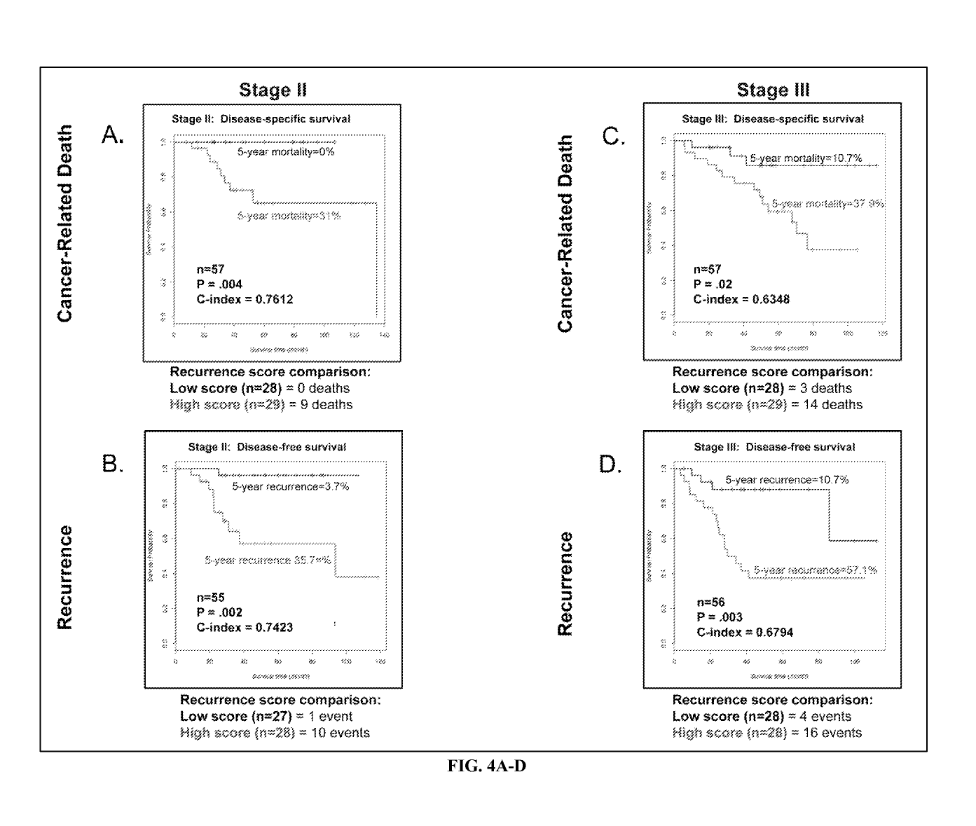

In order to determine if we could identify high-risk stage II and III patients, we tested the recurrence score on stage II and III patients in the MCC dataset. Low recurrence score patients in each group (stage II alone, FIGS. 4A-B and stage III alone, FIGS. 4C-D) demonstrated significantly better outcomes than high recurrence score patients. As can be seen in the figure, this finding held true in both analyses using the endpoints of disease-specific survival (cancer-related death, FIGS. 4A and 4C) as well as disease-free survival (recurrence, FIGS. 4B and 4D).

To determine if previously described, yet unproven, pathologic “high-risk” features in the stage II patients were associated with either the high or the low recurrence score we reviewed the available data regarding T4 lesions, lymph node retrieval and histology in the larger MCC dataset. In the disease-specific survival analysis, four of the 28 low recurrence score patients had T4 lesions, while only one of the 29 high recurrence score patients had a T4 lesion (p=0.19). In the stage II disease-free survival analysis, the four T4 lesions were evenly distributed between high (n=2) and low (n=2) recurrence score patients (p>0.99). The numbers of T4 tumors are small in the MCC test set and the inventors will need to assess these characteristics as the recurrence score is tested in a larger set of colon cancer patients before making any strong statements in association with the 34-gene recurrence score.

The inventors found no significant differences in the distribution of well-differentiated, moderately differentiated or poorly differentiated tumors in high or low score stage II patients (p=0.47 and 0.22 respectively). Furthermore, in regard to the number of lymph nodes retrieved and the recurrence score in the disease-specific survival analysis, the inventors found that 12 of 28 low score patients had less than 12 lymph nodes retrieved while 12 of 29 high score patients had less than 12 lymph nodes retrieved (p>0.99). The inventors also queried differentiation status and lymph nodes retrieved in the stage III patients and found no association with recurrence score and differentiation status or number of lymph nodes retrieved (data not shown). These data indicate that the recurrence score performs independently of traditional pathological markers.

Notably, there were no cancer-related deaths and only one recurrence event in low recurrence score stage II patients (FIGS. 4A and 4C). At five years, 31% of the high recurrence score stage II patients had died of cancer versus none of the low score patients. For the stage III patients, the five-year mortality rate was 10.7% for low recurrence score patients as compared with 37.9% for the high recurrence score patients (FIG. 4C). The median survival time for stage III patients with a high recurrence score was 29.4 months. In sharp contrast, the low recurrence score stage III patients as a group faired so well that none of the patients in this group have reached the threshold for calculating their median survival time. These data show that the 34-gene recurrence score can discriminate stage II and III colon cancer patients that have a low- or high-risk of cancer recurrence and death. A high recurrence score in univariate and multivariate analyses predicts recurrence and survival. The recurrence score was tested in the MCC patient dataset to determine the relative risk of recurrence and cancer-related death. Patients with a high recurrence score had increased relative risk of recurrence, as measured by hazard ratios (HR) across all stages (Table 3, HR 4.9, p<0.001). High score stage II patients were also at increased relative risk of recurrence (HR 13.1, p=0.01). Finally, the relative risk of recurrence in stage III patients with a high score was increased in this analysis (HR 4.7, p=0.006). These data show that the recurrence score based on the 34-gene signature is a strong predictor of recurrence and cancer-related death in colon cancer patients.

TABLE 3
The 34-gene recurrence score associates with increased
risk of recurrence

| | | | | Upper 95% |
|---|---|---|---|---|
| | Hazard ratio | p-value | Lower 95% CI | CI |
| Disease-free surv. | 4.9 | <0.001 | 2.157 | 11.27 |
| (all stages) | | | | |
| Disease-free surv. | 13.1 | 0.01 | 1.660 | 103.1 |
| (Stage II) | | | | |
| Disease-free surv. | 4.7 | 0.006 | 1.566 | 14.5 |
| (Stage III) | | | | |

Univariate analysis was done using the recurrence scores to segregate patients from the MCC data set into higher than median and lower than median score groups. Hazard ratios were calculated for each patient group related to disease-free survival. Hazard ratios in this analysis range from 4.9 across all stages to 4.7 for stage III patients. 95% confidence intervals and p-values are given in the table.

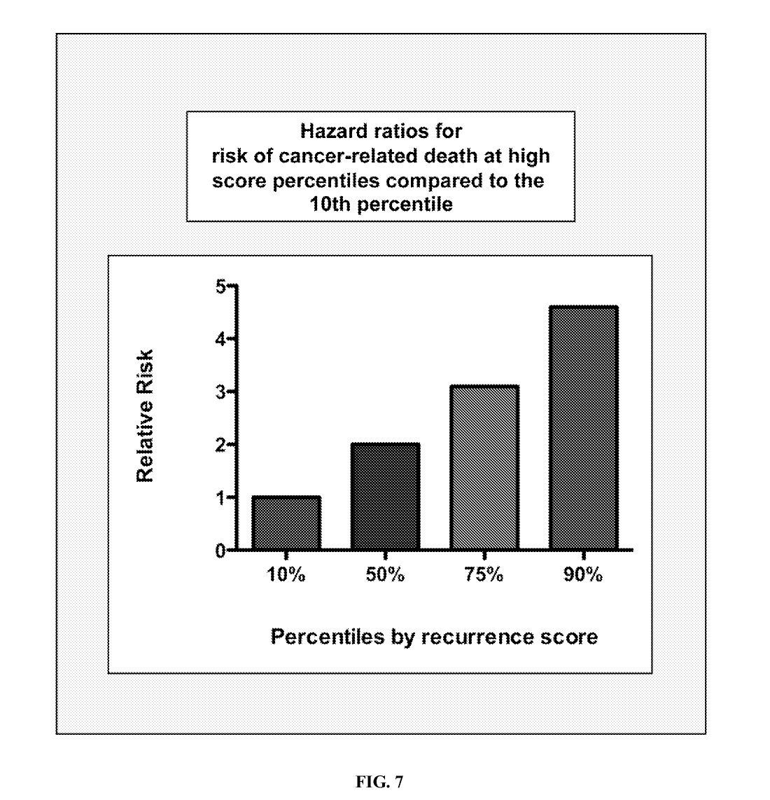

Multivariate analysis of the MCC patient data was performed to determine independent predictors of recurrence and survival. After adjusting for recurrence score, gender, tumor stage, age and tumor grade, only the recurrence score (p<0.001) and tumor stage (p=0.002) were significant determinants of cancer recurrence (Table 7). The inventors also used disease-specific survival as the outcome measure in univariate and multivariate models and observed similar results (Tables 8 and 9). Furthermore, the magnitude of the recurrence score was significantly associated with outcome. Hazard ratios for cancer-related death demonstrated that across all stages, high recurrence score patients in the 75th and 90th percentiles are at increased relative risk of cancer-related death (HR=3.1 and 4.6, respectively) compared with those patients with scores in the 10^{th} percentile (FIG. 7). Therefore, the 34-gene recurrence score is an independent predictor of cancer recurrence and death in colon cancer patients.

TABLE 7
Recurrence score is an independent predictor of recurrence risk

| | Adj. Hazard | | | |
|---|---|---|---|---|
| | ratio | p-value | Lower 95% CI | Upper 95% CI |
| Recurrence | 1.016 | <0.001 | 1.008 | 1.025 |
| score | | | | |
| Gender | 1.011 | 0.98 | 0.481 | 2.124 |
| Stage | 2.119 | 0.002 | 1.312 | 3.424 |
| Age | 1.001 | 0.93 | 0.974 | 1.029 |
| Grade | 1.446 | 0.32 | 0.701 | 2.985 |

A summary of a multivariate analysis, using the Moffitt Cancer Center patient variables to calculate independent risk factors, is shown in the table. Recurrence score (p < 0.001) and stage (p = 0.002) were each found to be significant predictors of disease-free survival in the multivariate model. Recurrence score, age, gender and grade were adjusted in the multivariate Cox model for disease-free survival and the results for each risk factor and disease-free survival are shown. The adjusted p-values as well as the adjusted 95% confidence intervals of the beta-coefficient from the Cox model were reported.

TABLE 8
Recurrence score associates with increased risk of cancer-related death

| | | | | Upper 95% |
|---|---|---|---|---|
| | Hazard ratio | p-value | Lower 95% CI | CI |
| Disease-free surv. | 4.4 | <0.001 | 2.25 | 8.45 |
| (all stages) | | | | |
| Disease-free surv. | NA | 0.003 | NA | NA |
| (Stage II) | | | | |
| Disease-free surv. | 4.1 | 0.03 | 1.17 | 14.2 |
| (Stage III) | | | | |

Univariate analysis using the recurrence score to segregate patients from the Moffitt Cancer Center data set into higher than median and lower than median score groups. Hazard ratios were calculated for each patient group related to disease-specific survival (DSS). Hazard ratios from 4.4 across all stages to 4.1 for stage III were noted in this analysis. 95% confidence intervals and p-values for the test given in the table.
NA implies that no hazard ratio from the Cox model was calculated as no cancer-related deaths occurred in the low score stage II patients. p-value for stage II DSS in this model was calculated according to exact log-rank test for unequal follow-up.

TABLE 9
Recurrence score is an independent predictor of cancer-related death

| | | | | Upper 95% |
|---|---|---|---|---|
| | Adj. Hazard ratio | p-value | Lower 95% CI | CI |
| Recurrence | 1.021 | <0.001 | 1.014 | 1.028 |
| score | | | | |
| Gender | 0.884 | 0.68 | 0.493 | 1.588 |
| Stage | 4.98 | <0.001 | 3.113 | 7.967 |
| Age | 1.026 | 0.04 | 1.002 | 1.051 |
| Grade | 1.61 | 0.12 | 0.887 | 2.922 |

A summary of a multivariate analysis, using the Moffitt Cancer Center patient variables to calculate independent risk factors, is shown in the table. Recurrence score (p < 0.001), stage (p < 0.001) and age (p = 0.04) were each found to be significant predictors of disease-specific survival (DSS) in the multivariate model. Recurrence score, age, gender and grade were adjusted in the multivariate Cox model for both overall and disease-specific survival and the results for each risk factor and DSS are shown. The adjusted p-values as well as the adjusted 95% confidence intervals of the beta-coefficient from the Cox model were reported.

The 34-gene based recurrence score is associated with patient benefit and adjuvant chemotherapy in stage III colon cancer patients. As described above, a significantly greater proportion of stage III patients with a high recurrence score died of cancer as compared with low recurrence score patients (Table 10, p=0.003). Thirty percent (17 of 57 patients) of the stage III patients did not receive adjuvant chemotherapy (CTX). Therefore, the inventors sought to determine if there was a difference in survival between high and low recurrence score patients related to CTX administration. Stage III patients with a low recurrence score had equivalent survival outcomes regardless of whether or not they received adjuvant CTX (10% with CTX versus 12.5% without CTX, p>0.99). Among stage III patients with a high recurrence score, a significantly greater proportion of patients who did not receive CTX died from their cancer as compared with those who did receive adjuvant CTX (86% vs. 36%, p=0.04). There was no statistically significant difference in follow-up interval or in the proportion of patients receiving CTX when comparing high and low recurrence score groups (p=0.576 and p=0.770). These data suggest that stage III patients with a low recurrence score did not gain significant benefit from adjuvant CTX, whereas stage III patients with a high recurrence score had a better outcome after adjuvant CTX.

TABLE 10
Recurrence score associates with patient benefit and adjuvant
chemotherapy in stage III colon cancer patients

| Recurrence score/survival | Alive | Cancer-related death | p-value |
|---|---|---|---|
| Low score | 25 (62.5%) | 3 (17.6%) | 0.003 |
| High score | 15 (37.5%) | 14 (82.4%) | |

| | CTX | No CTX | |
|---|---|---|---|
| Low score | (n = 20) | (n = 8) | p-value |
| Cancer-related death | 2 (10%) | 1 (12.5%) | >0.99 |
| Alive | 18 (90%) | 7 (87.5%) | |
| | CTX | No CTX | |
| High score | (n = 22) | (n = 7) | p-value |
| Cancer-related death | 8 (36.4%) | 6 (85.7%) | 0.04 |
| Alive | 14 (63.6%) | 1 (14.3%) | |

Table of proportions analyzing associations between recurrence score, cancer-related death and exposure to adjuvant chemotherapy in stage III patients from the MCC data set are depicted. As can be seen in the low score panel, no significant association was found between a low recurrence score and cancer-related death in those stage III patients receiving chemotherapy versus those who did not receive chemotherapy (Fisher's Exact Test).

### Example 3

### Discussion

In the present study, the biology of colon cancer metastasis was modeled in immunocompetent mice to develop a gene expression signature that discriminates recurrence and survival outcomes in human colon cancer patients. Stage II and stage III patients bearing primary colon cancers that reflected this metastasis gene expression pattern were at greater relative risk of recurrence than those who did not (hazard ratios of 13.1 and 4.7, respectively). This gene expression profile, tested with a recurrence scoring method, performed independently of conventional pathological staging.

Perhaps most importantly, this recurrence score identifies stage II patients at high risk of recurrence and death and stage III patients at low risk of recurrence and death. The inventors have identified a subset of high-risk stage II patients that that may benefit from adjuvant therapy and a subset of low-risk stage II patients who will have an excellent outcome after surgical resection without adjuvant therapy. The inventors found that the 5-year survival rate was >95% in stage II patients with a low recurrence score, suggesting that adjuvant chemotherapy would provide minimal benefit in this group of patients. In contrast, 31% of stage II patients with a high recurrence score died of cancer. Of the nine stage II patients with a high recurrence score who died of cancer in the MCC dataset, 7 did not receive adjuvant chemotherapy. The inventors' preliminary analyses of these data suggest that high recurrence score stage II patients should be further studied to determine whether they will benefit from adjuvant therapy.

A unique aspect of this study is the fortuitous inclusion of sufficient numbers of stage III patients who did not receive adjuvant chemotherapy in the MCC database. This enabled an evaluation of whether the molecular recurrence score could predict response to adjuvant therapy. Of the high recurrence score stage III patients who were treated with adjuvant chemotherapy only 36.4% died from cancer, whereas 85.7% of the high score patients who did not receive adjuvant chemotherapy died from cancer. Despite the small numbers in these sub-groups the differences were statistically significant. More importantly, equally low proportions of stage III patients with a low recurrence score died of cancer regardless of administration of chemotherapy. Our data suggest that there is a low-risk group of stage III patients who could be surgically cured and spared the morbidity, expense and potential mortality associated with adjuvant chemotherapy. This is consistent with prior observations from randomized clinical trials that established the benefits of adjuvant chemotherapy in stage III colon cancer where 40-44% of patients enrolled in the surgery-only groups did not recur in five years even without adjuvant treatment (Ragnhammar et al., 2001). Determination of an objective scoring method whereby the 34-gene profile can be tested in a prospective fashion is ongoing and will be required to determine if the 34-gene based recurrence score can be used clinically to guide decisions regarding adjuvant therapy for stage III colon cancer patients.

Several investigative groups have reported gene expression signatures with predictive power in breast, lung, liver and colorectal cancers (Barrier et al., 2006; Shedden et al., 2008; Paik et al., 2004; Hoshida et al., 2008; Wang et al., 2004; Lin et al., 2007). A 43-gene poor-prognosis prognosis signature for colorectal cancer provides a classifier for stage II and III patients as a molecular staging device (Eschrich et al., 2005). In a more recent study, a computational model was used to derive a 50-gene signature and a recurrence score for early stage colon cancer (Garman et al., 2008). There was no overlap between our 34-gene signature and the above-described 50-gene signature. This finding is not surprising since one model is computationally derived and the other is based on the biology of metastasis. The inventors find it interesting that 13 of the 34 genes in our proposed signature have previously described roles in cancer, and several others are involved in cell-cell signaling, immune response, cell proliferation, embryonic development and cell migration that will lead to mechanism-based hypothesis testing.

Although a high recurrence score based on the 34-gene metastasis signature worked well in this study, the number of significant genes reported was not based on the smallest number of genes that could discriminate the survival endpoint, but was based upon the combined statistical, biological and clinical evidence. There is also the possibility that some of the computationally derived genes discovered in human datasets would be missed in a mouse model; however, the biological basis of the 34-gene signature derived from our mouse model seems to be a robust predictor. The possibility of achieving similar or better survival discrimination with different subsets of the genes certainly exists; however, the inventors feel that the biological basis of our study provides a solid foundation for further translational application and testing of our model.

The cross-species functional genomics approach yields insights into the molecular mechanisms of the metastatic process. Consistent with our approach, gene expression patterns identified in wound healing have been applied successfully to breast cancer outcomes (Chang et al., 2004). In addition, cell culture and mouse models have also demonstrated relevance to clinical outcomes in hepatocellular carcinoma using gene expression profiling (Lee et al., 2004; Kaposi-Novak et al., 2006). Similarly, the inventors' approach has uncovered a gene signature with prognostic significance in colon cancer.

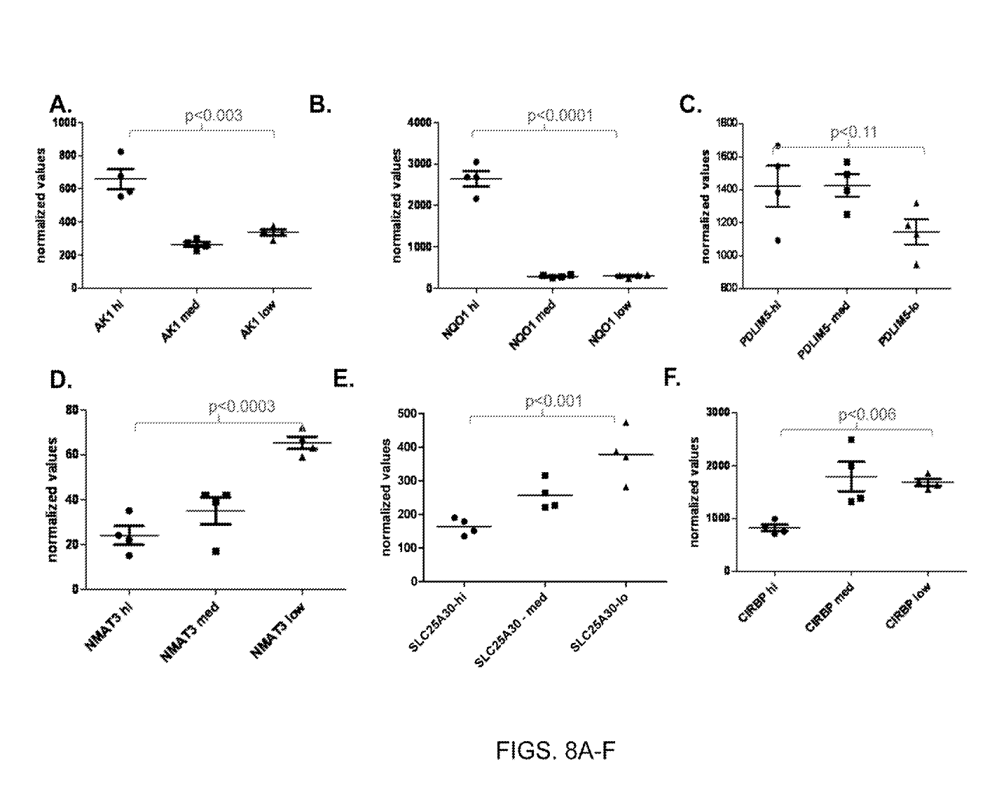

The inventors tested a multiplexed quantitative Nuclease Protection Assay (qNPA), commercially available and customizable through HTG technologies. For these studies, quantitative results were obtained from 3-5 micron slices of formalin fixed tissue per sample from colon cancer specimens with metastasis scores determined by microarray. In FIGS. 8A-F, differences are measured between the expression of 3 representative patients with high (“hi”, indicated by closed circles), medium (“med”, indicated by closed squares), or low (“lo” or “low”, indicated by closed triangles) metastasis scores. For each element, measures in arbitrary units were taken in quadruplicate, normalized to a housekeeping gene, and graphed with each normalized measure shown and mean values indicated with a horizontal bar. Thus, the data shows expression of 3 signature elements (AK1, NQO1, PDLIM5) known to be upregulated in patients with high metastasis score were predictably, measurably and significantly (AK 1, NQO 1) distinct between known high and low score patients by this assay. Similarly, expression of 3 signature elements (NMAT3, SLC25A30, CIRBP) known to be downregulated in patients with high metastasis score were predictably, measurably and significantly distinct between high and low score patients.

In conclusion, the 34-gene based recurrence score can identify stage II and III patients at greater risk of colon cancer recurrence and death. Again, this biologically-based expression signature identifies a potential method for more appropriate selection of patients for systemic therapy after curative-intent surgical resection of colon cancer. Future prospective studies are needed to confirm whether chemotherapy may be safely avoided in stage III patients with a low recurrence score and whether stage II patients with a high recurrence score can achieve a better outcome if they receive adjuvant chemotherapy.

TABLE 11
RANKED GENE LIST

| | | |
|---|---|---|
| 1 | MGP | Matrix Gla protein |
| 2 | PRTN3 | Proteinase 3 |
| 3 | TEX11 | Tstis expressed 11 |
| 4 | EGR1 | Erly growth response 1 |
| 5 | HS3ST5 | Hparan sulfate (glucosamine) 3-O-sulfotransferase 5 |
| 6 | SPRY4 | Sprouty homolog 4 (Drosophila) |
| 7 | SLC25A30 | Solute carrier family 25, member 30 |
| 8 | C6orf64 | Cromosome 6 open reading frame 64 |
| 9 | PDLIM5 | PDZ and LIM domain 5 |
| 10 | AK1 | Adenylate kinase |
| 11 | DFNB31 | Deafness, autosomal recessive 31 |
| 12 | DCTD | dCMP deaminase |
| 13 | SYT17 | Synaptoagmin XVII |
| 14 | CSN3 | Casein kappa |
| 15 | CXR7 | Chemokine (C—X—C) receptor 7 |
| 16 | TACC2 | Transforming, acidid coiled-coil containing protein 2 |
| 17 | ACTB | Actin β |
| 18 | DENND2A | DENN/MADD domain containing 2A |
| 19 | ACPY2 | Acylphosphatase 2, muscle type |
| 20 | MMP13 | Matrix metalloproteinase 13 (collagenase 3) |
| 21 | S100A3 | S100 calcium binding protein A3 |
| 22 | HES1 | Hairy and enhancer split 1 (Drosophila) |
| 23 | VDR | Vitamin D (1,25-dihydroxyvitamin D3) receptor |
| 24 | SPDYA | Speedy homology A (Xenopus laevis) |
| 25 | CIRBP | Cold inducible RNA binding protein |
| 26 | TMEM14A | Transmembrane protein 14A |
| 27 | HPSE | Heparanase |
| 28 | NQ01 | NAD(P)H dehydrogenase, quinone 1 |
| 29 | MUM1L1 | Melanoma associated antigen (mutated) 1-like 1 |
| 30 | MYOT | Myotilin |
| 31 | C20orf74 | Chromosome 20 open reading frame 74 |
| 32 | STOX2 | Storkhead box 2 |
| 33 | CRABP1 | Cellular retinoic acid binding protein 1 |
| 34 | NMNAT3 | Nicotinamide nucleotice acetyltransferase 3 |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,196,265
U.S. Pat. No. 4,275,149
U.S. Pat. No. 4,277,437
U.S. Pat. No. 4,366,241
U.S. Pat. No. 4,415,723
U.S. Pat. No. 4,458,066
U.S. Pat. No. 4,683,195
U.S. Pat. No. 4,683,202
U.S. Pat. No. 4,684,611
U.S. Pat. No. 4,800,159
U.S. Pat. No. 4,879,236
U.S. Pat. No. 4,883,750
U.S. Pat. No. 4,952,500
U.S. Pat. No. 5,143,854
U.S. Pat. No. 5,202,231
U.S. Pat. No. 5,242,974
U.S. Pat. No. 5,279,721
U.S. Pat. No. 5,288,644
U.S. Pat. No. 5,302,523
U.S. Pat. No. 5,322,783
U.S. Pat. No. 5,324,633
U.S. Pat. No. 5,384,253
U.S. Pat. No. 5,384,261
U.S. Pat. No. 5,405,783
U.S. Pat. No. 5,412,087
U.S. Pat. No. 5,424,186
U.S. Pat. No. 5,429,807
U.S. Pat. No. 5,432,049
U.S. Pat. No. 5,436,327
U.S. Pat. No. 5,445,934
U.S. Pat. No. 5,464,765
U.S. Pat. No. 5,468,613
U.S. Pat. No. 5,470,710
U.S. Pat. No. 5,472,672
U.S. Pat. No. 5,492,806
U.S. Pat. No. 5,503,980
U.S. Pat. No. 5,510,270
U.S. Pat. No. 5,525,464
U.S. Pat. No. 5,527,681
U.S. Pat. No. 5,529,756
U.S. Pat. No. 5,532,128
U.S. Pat. No. 5,538,877
U.S. Pat. No. 5,538,880
U.S. Pat. No. 5,545,531
U.S. Pat. No. 5,547,839
U.S. Pat. No. 5,550,318
U.S. Pat. No. 5,554,501
U.S. Pat. No. 5,556,752
U.S. Pat. No. 5,561,071
U.S. Pat. No. 5,563,055
U.S. Pat. No. 5,571,639
U.S. Pat. No. 5,580,726
U.S. Pat. No. 5,580,732
U.S. Pat. No. 5,580,859
U.S. Pat. No. 5,589,466
U.S. Pat. No. 5,591,616
U.S. Pat. No. 5,593,839
U.S. Pat. No. 5,599,672
U.S. Pat. No. 5,599,695
U.S. Pat. No. 5,610,042
U.S. Pat. No. 5,610;287
U.S. Pat. No. 5,624,711
U.S. Pat. No. 5,631,134
U.S. Pat. No. 5,639,603
U.S. Pat. No. 5,654,413
U.S. Pat. No. 5,656,610
U.S. Pat. No. 5,658,734
U.S. Pat. No. 5,661,028
U.S. Pat. No. 5,665,547
U.S. Pat. No. 5,667,972
U.S. Pat. No. 5,693,762
U.S. Pat. No. 5,695,940
U.S. Pat. No. 5,700,637
U.S. Pat. No. 5,702,932
U.S. Pat. No. 5,736,524
U.S. Pat. No. 5,739,169
U.S. Pat. No. 5,744,305
U.S. Pat. No. 5,780,448
U.S. Pat. No. 5,789,215
U.S. Pat. No. 5,795,715
U.S. Pat. No. 5,800,992
U.S. Pat. No. 5,801,005
U.S. Pat. No. 5,807,522
U.S. Pat. No. 5,824,311
U.S. Pat. No. 5,830,645
U.S. Pat. No. 5,830,880
U.S. Pat. No. 5,837,196
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,846,945
U.S. Pat. No. 5,847,219
U.S. Pat. No. 5,849,481
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,155
U.S. Pat. No. 5,861,244
U.S. Pat. No. 5,863,732
U.S. Pat. No. 5,863,753
U.S. Pat. No. 5,866,331
U.S. Pat. No. 5,866,366
U.S. Pat. No. 5,871,928
U.S. Pat. No. 5,871,986
U.S. Pat. No. 5,876,932
U.S. Pat. No. 5,882,864
U.S. Pat. No. 5,889,136
U.S. Pat. No. 5,900,481
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,626
U.S. Pat. No. 5,919,626
U.S. Pat. No. 5,919,630
U.S. Pat. No. 5,922,574
U.S. Pat. No. 5,925,517
U.S. Pat. No. 5,925,565
U.S. Pat. No. 5,928,862
U.S. Pat. No. 5,928,869
U.S. Pat. No. 5,928,905
U.S. Pat. No. 5,928,906
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,819
U.S. Pat. No. 5,935,825
U.S. Pat. No. 5,939,291
U.S. Pat. No. 5,942,391
U.S. Pat. No. 5,945,100
U.S. Pat. No. 5,981,274
U.S. Pat. No. 5,994,624
U.S. Pat. No. 6,004,755
U.S. Pat. No. 6,020,192
U.S. Pat. No. 6,054,297
U.S. Pat. No. 6,087,102
U.S. Pat. No. 6,368,799
U.S. Pat. No. 6,383,749
U.S. Pat. No. 6,506,559
U.S. Pat. No. 6,573,099
U.S. Pat. No. 6,617,112
U.S. Pat. No. 6,638,717
U.S. Pat. No. 6,720,138
U.S. Patent Publn. 2002/0168707
U.S. Patent Publn. 2003/0051263
U.S. Patent Publn. 2003/0055020
U.S. Patent Publn. 2003/0159161
U.S. Patent Publn. 2004/0064842
U.S. Patent Publn. 2004/0265839
U.S. Patent Publn. 2008/0009439
Abbondanzo et al., Breast Cancer Res. Treat., 16:182(151), 1990.
Allred et al., Arch. Surg., 125(1):107-113, 1990.
Almendro et al., J. Immunol., 157(12):5411-5421, 1996.
Arap et al., Cancer Res., 55(6):1351-1354, 1995.
Austin-Ward and Villaseca, Revista Medica de Chile, 126(7):838-845, 1998.
Ausubel et al., In: Current Protocols in Molecular Biology, John, Wiley & Sons, Inc, NY, 1994.
Baichwal and Sugden, In: Gene Transfer, Kucherlapati (Ed.), Plenum Press, NY, 117-148, 1986.
Bakhshi et al., Cell, 41(3):899-906, 1985.
Barrier et al., J. Clin. Oncol., 24:4685-4691, 2006.
Beer et al., Nat. Med., 8:816-824, 2002.
Bellus, J. Macromol. Sci. Pure Appl. Chem., A31(1): 1355-1376, 1994.
Benson et al., J. Clin. Oncol., 22:3408-3419, 2004.
Bosher and Labouesse, Nat. Cell. Biol., 2(2):E31-E36, 2000.
Brown et al. Immunol. Ser., 53:69-82, 1990.
Brummelkamp et al., Cancer Cell, 2:243-247, 2002.
Brummelkamp et al., Science, 296(5567):550-553, 2002.
Bukowski et al., Clinical Cancer Res., 4(10):2337-2347, 1998.
Caldas et al., Cancer Res., 54:3568-3573, 1994.
Caldas et al., Nat. Genet., 8(1):27-32, 1994.
Capaldi et al., Biochem. Biophys. Res. Comm., 74(2):425-433, 1977.
Carbonelli et al., FEMS Microbiol. Lett., 177(1):75-82, 1999.
Chandler et al., Proc. Natl. Acad. Sci. USA, 94(8):3596-601, 1997.
Chang et al., PLoS Biol., 2:E7, 2004.
Chen and Okayama, Mol. Cell Biol., 7(8):2745-2752, 1987.
Cheng et al., Cancer Res., 54(21):5547-5551, 1994.
Christodoulides et al., Microbiology, 144(Pt 11):3027-3037, 1998.
Cleary and Sklar, Proc. Natl. Acad. Sci. USA, 82(21):7439-7443, 1985.
Cleary et al., J. Exp. Med., 164(1):315-320, 1986.
Cocea, Biotechniques, 23(5):814-816, 1997.
Coupar et al., Gene, 68:1-10, 1988.
Davidson et al., J. Immunother., 21(5):389-398, 1998.
De Jager et al., Semin. Nucl. Med., 23(2):165-179, 1993.
Doolittle and Ben-Zeev, Methods Mol, Biol., 109:215-237, 1999.
Eschrich et al., J. Clin. Oncol., 23:3526-3535, 2005.
European Appln. 320 308
European Appln. 329 822
European Appln. 373 203
European Appln. 785 280
European Appln. 799 897
Fechheimer et al., Proc Natl. Acad. Sci. USA, 84:8463-8467, 1987.
Figueredo et al., Cochrane Database Syst Rev., CD005390, 2008.
Figueredo et al., J. Clin. Oncol., 22:3395-3407, 2004.
Fire et al., Nature, 391(6669):806-811, 1998.
Fodor et al., Science, 251:767-773, 1991.
Fraley et al., Proc. Natl. Acad. Sci. USA, 76:3348-3352, 1979.
Friedmann, Science, 244:1275-1281, 1989.
Frohman, In: PCR Protocols: A Guide To Methods And Applications, Academic Press, N.Y., 1990.
Garman et al., Proc. Natl. Acad. Sci. USA, 105:19432-19437, 2008.
GB Application No. 2 202 328
Gill et al., J. Clin. Oncol., 22:1797-1806, 2004.
Graham and Van Der Eb, Virology, 52:456-467, 1973.
Grishok et al., Science, 287:2494-2497, 2000.
Gulbis and Galand, Hum. Pathol., 24(12):1271-1285, 1993.
Hacia et al., Nature Genet., 14:441-449, 1996.
Hanibuchi et al., Int. J. Cancer, 78(4):480-485, 1998.
Harland and Weintraub, J. Cell Biol., 101(3):1094-1099, 1985.
Harlow and Lane, In: Antibodies: A Laboratory Manual, Cold Spring Harbor Laboratory, Cold Spring Harbor, N.Y., 346-348, 1988.
Harrell et al., JAMA, 247:2543-2546, 1982.
Harrell et al., JAMA, 247:2543-2546, 1982.
Hedenfalk et al., N. Engl. J. Med., 344:539-548, 2001.
Heinze et al., Biometrics, 59:1151-1157, 2003.
Hellstrand et al., Acta Oncologica, 37(4):347-353, 1998.
Hermonat and Muzycska, Proc. Natl. Acad. Sci. USA, 81:6466-6470, 1984.
Horwich et al. J. Virol., 64:642-650, 1990.
Hoshida et al., N. Engl. J. Med., 359:1995-2004, 2008.
Hui and Hashimoto, Infection Immun., 66(11):5329-5336, 1998.
Hussussian et al., Nat. Genet., 8(1):15-21, 1994.
Innis et al., Proc. Natl. Acad. Sci. USA, 85(24):9436-9440, 1988.
Inouye and Inouye, Nucleic Acids Res., 13:3101-3109, 1985.
Irizarry et al., Biostatistics, 4:249-64, 2003.
Jemal et al., CA Cancer J. Clin., 58:71-96, 2008.
Jenkins et al., Clin. Exp. Metastasis, 20:733-44, 2003.
Ju et al., Gene Ther., 7(19):1672-1679, 2000.
Kaeppler et al., Plant Cell Rep., 8:415-418, 1990.
Kamb et al., Nat. Genet., 8(1):23-26, 1994.
Kamb et al., Science, 2674:436-440, 1994.
Kaneda et al., Science, 243:375-378, 1989.
Kaposi-Novak et al., J. Clin. Invest., 116:1582-1595, 2006.
Kato et al, J. Biol. Chem., 266:3361-3364, 1991.
Kerr et al., Br. J. Cancer, 26(4):239-257, 1972.
Ketting et al., Cell, 99(2):133-141, 1999.
Kraus et al. FEBS Lett., 428(3):165-170, 1998.
Kwoh et al., Proc. Natl. Acad. Sci. USA, 86:1173, 1989.
Lafreniere and Rosenberg, J. Natl. Cancer Inst., 76:309-322, 1986.
Lareyre et al., J. Biol. Chem., 274(12):8282-8290, 1999.
Lee and Thorgeirsson, Gastroenterology, 127:S51-55, 2004.
Lee et al., Biochem. Biophys. Res. Commun., 238(2):462-467, 1997.
Lee et al., Biochim. Biophys. Acta, 1582:175-177, 2002.
Lee et al., Nat. Genet., 36:1306-1311, 2004.
Levenson et al., Hum. Gene Ther., 9(8):1233-1236, 1998.
Lin and Avery, Nature, 402:128-129, 1999.
Lin et al., Clin. Cancer Res., 13:498-507, 2007.
MacBeath and Schreiber, Science, 289(5485):1760-1763, 2000.
Macejak and Sarnow, Nature, 353:90-94, 1991.
Mamounas et al., J. Clin. Oncol., 17:1349-1355, 1999.
Mitchell et al., Ann. NY Acad. Sci., 690:153-166, 1993.
Mitchell et al., J. Clin. Oncol., 8(5):856-869, 1990.
Montgomery et al., Proc. Natl. Acad. Sci. USA, 95:15502-15507, 1998.
Mori et al., Cancer Res., 54(13):3396-3397, 1994.
Morton et al., Arch. Surg., 127:392-399, 1992.
Nakamura et al., In: Handbook of Experimental Immunology (4^{th} Ed.), Weir et al. (Eds), 1:27, Blackwell Scientific Publ., Oxford, 1987.
Nicolas and Rubinstein, In: Vectors: A survey of molecular cloning vectors and their uses, Rodriguez and Denhardt, eds., Stoneham: Butterworth, pp. 494-513, 1988.
Nicolau and Sene, Biochim. Biophys. Acta, 721:185-190, 1982.
Nicolau et al., Methods Enzymol., 149:157-176, 1987.
Nobori et al., Nature (London), 368:753-756, 1995.
Nomoto et al., Gene, 236(2):259-271, 1999.
Ohara et al., Proc. Natl. Acad. Sci. USA, 86:5673-5677, 1989.
Okamoto et al., Proc. Natl. Acad. Sci. USA, 91(23):11045-11049, 1994.
Omirulleh et al., Plant Mol. Biol., 21(3):415-428, 1993.
Orlow et al., Cancer Res, 54(11):2848-2851, 1994.
Paik et al., N. Engl. J. Med., 351:2817-2826, 2004.
Pandey and Mann, Nature, 405(6788):837-846, 2000.
Paul et al., Nature Biotechnol., 20:505-508, 2002.
PCT Appln. PCT/US87/00880
PCT Appln. PCT/US89/01025
PCT Appln. WO 00/44914
PCT Appln. WO 01/36646
PCT Appln. WO 01/68836
PCT Appln. WO 0138580
PCT Appln. WO 0168255
PCT Appln. WO 03020898
PCT Appln. WO 03022421
PCT Appln. WO 03023058
PCT Appln. WO 03029485
PCT Appln. WO 03040410
PCT Appln. WO 03053586
PCT Appln. WO 03066906
PCT Appln. WO 03067217
PCT Appln. WO 03076928
PCT Appln. WO 03087297
PCT Appln. WO 03091426
PCT Appln. WO 03093810
PCT Appln. WO 03100012
PCT Appln. WO 03100448A1
PCT Appln. WO 04020085
PCT Appln. WO 04027093
PCT Appln. WO 09923256
PCT Appln. WO 09936760
PCT Appln. WO 2004/048933
PCT Appln. WO 88/10315
PCT Appln. WO 89/06700
PCT Appln. WO 90/07641
PCT Appln. WO 93/17126
PCT Appln. WO 94/09699
PCT Appln. WO 95/06128
PCT Appln. WO 95/11995
PCT Appln. WO 95/21265
PCT Appln. WO 95/21944
PCT Appln. WO 95/35505
PCT Appln. WO 96/31622
PCT Appln. WO 97/10365
PCT Appln. WO 97/27317
PCT Appln. WO 9743450
PCT Appln. WO 99/32619
PCT Appln. WO 99/35505
Pease et al., Proc. Natl. Acad. Sci. USA, 91:5022-5026, 1994.
Pelletier and Sonenberg, Nature, 334(6180):320-325, 1988.
Pietras et al., Oncogene, 17(17):2235-2249, 1998.
Poste et al., Proc. Natl. Acad. Sci. USA, 78:6226-6230, 1981.
Potrykus et al., Mol. Gen. Genet., 199(2):169-177, 1985.
Qin et al., Proc. Natl. Acad. Sci. USA, 95(24):14411-14416, 1998.
Quasar Collaborative et al., Lancet., 370:2020-2029, 2007.
Ragnhammar et al., Acta Oncol., 40:282-308, 2001.
Ravindranath and Morton, Intern. Rev. Immunol., 7: 303-329, 1991.
Ridgeway, In: Vectors: A Survey of Molecular Cloning Vectors and Their Uses, Rodriguez et al. (Eds.), Stoneham: Butterworth, 467-492, 1988.
Rippe, et al., Mol. Cell Biol., 10:689-695, 1990.
Rosenberg et al., Ann. Surg. 210(4):474-548, 1989.
Rosenberg et al., N. Engl. J. Med., 319:1676, 1988.
Sambrook and Russell, Molecular Cloning: A Laboratory Manual 3^{rd} Ed., Cold Spring Harbor Laboratory Press, 2001.
Sambrook et al., In: Molecular cloning: a laboratory manual, 2^{nd} Ed., Cold Spring Harbor Laboratory Press, Cold Spring Harbor, N.Y., 1989.
Serrano et al., Nature, 366:704-707, 1993.
Serrano et al., Science, 267(5195):249-252, 1995.
Sharp and Zamore, Science, 287:2431-2433, 2000.
Sharp, Genes Dev., 13:139-141, 1999.
Shedden et al., Nat. Med., 14:822-827, 2008.
Shoemaker et al., Nature Genetics, 14:450-456, 1996.
Smyth et al., Bioinformatics, 21:2067-2075, 2005.
Sui et al., Proc. Natl. Acad. Sci. USA, 99(8):5515-5520, 2002.
Tabara et al., Cell, 99(2):123-132, 1999.
Temin, In: Gene Transfer, Kucherlapati (Ed.), NY, Plenum Press, 149-188, 1986.
Tsujimoto and Croce, Proc. Natl. Acad. Sci. USA, 83(14):5214-5218, 1986.
Tsujimoto et al., Nature, 315:340-343, 1985.
Tsumaki et al., J. Biol. Chem., 273(36):22861-22864, 1998.
Tukey, Control Clin. Trials, 14:266-285, 1993.
UK Appln. 8 803 000
Walker et al., Nucleic Acids Res. 20(7):1691-1696, 1992.
Wang et al., J. Clin. Oncol., 22:1564-1571, 2004.
Wincott et al., Nucleic Acids Res., 23(14):2677-2684, 1995.
Wong et al., Gene, 10:87-94, 1980.
Wu et al., Biochem. Biophys. Res. Commun., 233(1):221-226, 1997.
Wu et al., Mol. Ther. 4:297-306, 2001.
Yanagisawa et al., Lancet, 362:433-439, 2003.
Yu et al., J. Am. Chem. Soc., 124(23):6576-6583, 2002.
Zhao-Emonet et al., Biochim. Biophys. Acta, 1442(2-3):109-119, 1998.

What is claimed is:

1. A method of predicting prognosis in a human subject diagnosed with colorectal cancer comprising assessing expression of 3 or more of the following genes in a colorectal cancer sample obtained from said subject:
CXCR7, AK1, ACTB, MGP, HES1, TMEM14A, EGR1, VDR, C6orf64, NQO1, STOX2, ACPY2, SPRY4, DCTD, TACC2, PDLIM5, CRABP1, MMP13, MYOT, DFNB31, HPSE, TEX11, SYT17, MUM1L1, SLC25A30, CSN3, NMNAT3, DENND2A, CIRBP, SPDYA, S100A3, PRTN3, C20orf74 and HS3ST5;
wherein increased expression of at least 2-fold of CXCR7, AK1, ACTB, MGP, HES1, TMEM14A, EGR1, VDR, C6orf64, NQO1, STOX2, ACPY2, SPRY4, DCTD, TACC2 or PDLIM5 as compared to expression observed in non-cancer cells, and/or decreased expression of at least 2-fold of CRABP1, MMP13, MYOT, DFNB31, HPSE, TEX11, SYT17, MUM1L1, SLC25A30, CSN3, NMNAT3, DENND2A, CIRBP, SPDYA, S100A3, PRTN3, C20orf74 or HS3ST5 as compared to expression observed in non-cancer cells, indicates a poor prognosis.

2. The method of claim 1, wherein said colorectal cancer is stage I, II or III.

3. The method of claim 1, wherein expression of at least one of MGP, PRTN3, TEX11, EGR1, HS3ST5, SPRY4, SLC25A30, C6orf64, PDLIM5, AK1 or DFNB31 are analyzed.

4. The method of claim 1, wherein assessing expression comprises assessing protein expression.

5. The method of claim 4, wherein assessing protein expression comprises ELISA, RIA, immunohistochemistry, or mass spectrometry.

6. The method of claim 1, wherein assessing expression comprises assessing mRNA expression.

7. The method of claim 6, wherein assessing mRNA expression comprises quantitative RT-PCR, gene chip array expression, or Northern blotting.

8. The method of claim 1, wherein said expression observed in said non-cancer cell is a pre-determined standard.

9. The method of claim 1, wherein said expression observed in said non-cancer cell is determined by assessing expression in a non-cancer cell from said subject.

10. The method of claim 1, further comprising obtaining said colorectal cancer sample.

11. The method of claim 1, wherein said colorectal cancer is colon cancer.

12. The method of claim 1, wherein said colorectal cancer is rectal cancer.

13. The method of claim 1, wherein prognosis is length of survival.

14. The method of claim 13, wherein length of survival is disease-specific length of survival.

15. The method of claim 13, wherein length of survival is overall survival.

16. The method of claim 1, wherein prognosis is length of time to recurrence.

17. The method of claim 1, further comprising making a treatment decision for said subject.

18. The method of claim 17, wherein said treatment decision is to give chemotherapy to a subject having a poor prognosis as compared to median.

19. The method of claim 17, wherein said treatment decision is to not give chemotherapy to a subject having a favorable prognosis as compared to median.

20. The method of claim 18, further comprising treating said subject with adjuvant chemotherapy.

21. The method of claim 1, wherein at least 4 markers are analyzed.

22. The method of claim 1, wherein at least 5 markers are analyzed.

23. The method of claim 1, wherein at least 6 markers are analyzed.

24. The method of claim 1, wherein at least 7 markers are analyzed.

25. The method of claim 1, wherein at least 8 markers are analyzed.

26. The method of claim 1, wherein at least 9 markers are analyzed.

27. The method of claim 1, wherein at least 10 markers are analyzed.

28. The method of claim 1, wherein at least 11 markers are analyzed.

29. The method of claim 1, wherein at least 12 markers are analyzed.

30. The method of claim 1, wherein at least 13 markers are analyzed.

31. The method of claim 1, wherein at least 14 markers are analyzed.

32. The method of claim 1, wherein at least 15 markers are analyzed.

33. The method of claim 1, wherein 20 of said genes are analyzed.

34. The method of claim 1, wherein 25 of said genes are analyzed.

35. The method of claim 1, wherein 30 of said genes are analyzed.

36. The method of claim 1, wherein all 34 of said genes are analyzed.

37. The method of claim 1, wherein MGP, PRTN3 and TEX11 are analyzed.

38. The method of claim 37 further comprising analyzing EGR1.

39. The method of claim 38 further comprising analyzing HS3ST5.

40. The method of claim 39 further comprising analyzing SPRY4.

41. The method of claim 40 further comprising analyzing SLC25A30.

42. The method of claim 41, further comprising analyzing C6org64.

43. The method of claim 42, further comprising analyzing PDLIM5.

44. The method of claim 43, further comprising analyzing AK1.

45. The method of claim 44, further comprising analyzing DFNB31.
